(19) United States
(12) Patent Application Publication
(10) Pub. No.: US 2014/0120105 A1
(43) Pub. Date: May 1, 2014

(54) THERAPEUTIC TARGETING OF FICOLIN-3

(71) Applicants: Peter Garred, Charlottenlund (DK); Tina Hummelshoj Glue, Malov (DK)

(72) Inventors: Peter Garred, Charlottenlund (DK); Tina Hummelshoj Glue, Malov (DK)

(73) Assignees: KOBENHAVNS UNIVERSITET, Copenhagen K (DK); RIGSHOSPITALET, Copenhagen O (DK)

(21) Appl. No.: 14/127,295

(22) PCT Filed: Jun. 28, 2012
(86) PCT No.: PCT/DK2012/050216

§ 371 Date: Dec. 18, 2013

Related U.S. Application Data
(60) Provisional application No. 61/502,127, filed on Jun. 28, 2011.

(30) Foreign Application Priority Data
Jun. 28, 2011 (EP) 11171711.2

(51) Int. Cl. C07K 16/18

(52) U.S. Cl. CPC C07K 16/18
USPC 424/139.1; 530/387.9; 530/387.3

(57) ABSTRACT

The present Invention relates to novel antibodies against Ficolin-3, which antibodies inhibit complement activation. The invention further relates to the use of anti-Ficolin-3 antibodies in the treatment of conditions associated with inflammation, apoptosis, autoimmunity, coagulation, thrombotic or coagulopathic related diseases, as well as the use as biomarkers. The present invention further relates to nucleic acid molecules encoding such antibodies, vectors and host cells used in the production of the antibodies.

### FIELD OF THE INVENTION

The present invention relates to novel antibodies against Ficolin-3 (a.k.a. H-ficolin or Hakata antigen) derived from the FCN3 gene as well as all other substances that inhibit the binding of Ficolin-3 to ligands and downstream effector functions such as complement activation and induction of phagocytosis and inflammatory processes. The invention further relates to the use of anti-Ficolin-3 antibodies and other inhibitory substances in the treatment of conditions associated with inflammation, apoptosis, allograft rejection, autoimmunity, coagulation, thrombotic or coagulopathic related diseases, metabolic diseases and cancer as well as the use as biomarkers. The present invention further relates to nucleic acid molecules encoding such antibodies, vectors and host cells used in the production of the antibodies.

### BACKGROUND OF THE INVENTION

The complement system is an integral part of the innate immune system that protects the host against invading pathogens. Three distinct pathways constitute the complement system; the classical pathway, the alternative pathway and the lectin pathway. The C1 complex initiates the classical pathway upon recognition of immune complexes and dying host cells. The alternative pathway is spontaneously activated by C3 hydrolysis, but it has also been reported that properdin, a stabilizer of the alternative pathway convertase, is capable of initiating the complement cascade. The ficolins, collectin-11 and mannose-binding lectin (MBL) in association with MBL/Ficolin-associated serine proteases (MASPs) are the initiator molecules of the lectin pathway. Three MASPs (−1, −2 and −3) have been described so far and the current notion is that MASP-2 is the main lectin pathway activator. Upon recognition of pathogen-associated molecular patterns or altered self by MBL and the ficolins, the associated proteases cleave C4 and C2, hereby activating the complement cascade, which ultimately leads to the formation of the terminal complement complex (TCC). Moreover, the activation of MASPs has been shown to initiate activation of different components of the coagulation cascade and cellular receptors. Nevertheless, inappropriate and uncontrolled activation of the complement and coagulation systems has been associated with numerous diseases and is regarded to be a key component in adverse outcome of many inflammatory conditions. This is based both on in vitro and in vivo data. Thus inhibition of the different stages of the complement and coagulation activation pathways is a major target for the pharmaceutical industry. Ficolin-3 is the predominant plasma protein in the lectin pathway of complement and has been shown to have the strongest complement activating capacity among the initiators of the lectin pathway of complement. Its binding specificities are poorly defined, but it has been ample documented that it bind to and sequester apoptotic and necrotic cell material as well as certain endogenous ligands like acetylated albumin. Several studies suggest that deposition of ficolin-3 in tissues may be associated with nephropathy and preeclampsia. Thus it is reasonable to believe that ficolin-3 may initiate adverse inflammatory reactions.

Human Ficolin-3 (fibrinogen/collagen-like), also called H-ficolin and, previously Hakata antigen is a member of the ficolin family of secreted pattern recognition proteins that belong to the lectin complement activation pathway. Ficolin-3 is expressed by bile duct epithelial cells and hepatocytes, and is released into the bile and circulation, where it averages 25 μg/ml. It is also secreted by bronchial and alveolar epithelial cells in the lung. Mature human Ficolin-3 shares 46% and 52% amino acid (aa) identity with human Ficolin-1 and Ficolin-2, respectively. Ficolin-3 has been identified in primates and in lower species, but in some species like in rodents the ficolin-3 gene is described as a pseudogene and, which probably also is the situation in some porcine species like the domestic pig. The 35 kDa, 299 aa human Ficolin-3 (isoform 1) contains a signal sequence, an N-terminal collagen domain and a C-terminal fibrinogen like domain that includes a calcium binding site and two potential N-glycosylation sites. Isoform 2 is a variant lacking the 11 aa of exon 4 between the collagen and fibrinogen-like domains. The collagen domain mediates trimer formation. Ficolin-3 binds a set of carbohydrates containing N-acetylated glucosamine and galactosamine (GlcNAc and GalNAc) and glycine (GlyNAc), galactose or D-fucose as well as acetylated proteins. Binding of microbial carbohydrates initiates an immune response involving a calcium-dependent interaction of Ficolin-3 with the MBL/ficolin associated serine proteases (MASPs) complexes. This cleaves C4 to activate the complement pathway.

### OBJECT OF THE INVENTION

It is an object of embodiments of the invention to provide antibodies and other inhibitory molecules against Ficolin-3 suitable for the treatment of conditions associated with inflammation, apoptosis, autoimmunity, endocrine, coagulation, and/or thrombotic or coagulopathic related diseases. The antibodies of the invention may further be suitable as biomarkers for the diagnosis and/or prognosis of these indications as well as for malignant diseases, such as cancers.

### SUMMARY OF THE INVENTION

It has been found by the present inventor(s) that certain antibodies against Ficolin-3 inhibits the complement pathway of the immune system and accordingly therefore may be used in the treatment of specific medical conditions associated with inflammation, apoptosis, autoimmunity, coagulation, and/or thrombotic or coagulopathic related diseases.

Accordingly the present invention provides isolated anti-human Ficolin-3 monoclonal antibodies useful for therapeutic applications in humans. Typically, the antibodies are fully human or humanized to minimize the risk for immune responses against the antibodies when administered to a patient. As described herein, other antigen-binding molecules such as, e.g., antigen-binding antibody fragments, antibody derivatives, and multi-specific molecules, can be designed or derived from such antibodies. In one aspect, the antibodies are characterized by one or more functional properties, or by a combination of functional properties. Exemplary properties include, e.g., competing with at least one natural human Ficolin-3 ligand, or with several ligands, in binding to human Ficolin-3; reducing the amount of human Ficolin-3 in human plasma; binding of only one antibody molecule per human Ficolin-3; and/or binding to human Ficolin-3 with a dissociation constant (KD) of 10 nM or less. Certain anti-human Ficolin-3 antibodies of the invention may also or alternatively compete with, bind to essentially the same epitope as, or bind with the same or higher affinity as, one or more particular human anti-human Ficolin-3 antibody described herein, including anti-Ficolin-3 antibody FCN308, FCN334, and FCN329 described herein. For example, in some embodiments, the antibodies are also or alternatively more capable of competing with or blocking human Ficolin-3-binding of anti-Ficolin-3 antibody FCN308, FCN334, and FCN329 described herein than potentially known anti-human Ficolin-3 antibodies. In one embodiment, the antibodies bind to the same human Ficolin-3 epitope as anti-Ficolin-3 antibody FCN308, FCN334, and FCN329 described herein. In another embodiment, the antibodies also or alternatively bind the same epitope as anti-Ficolin-3 antibody FCN308, FCN334, and FCN329 described herein. In another aspect, the antibodies also or alternatively comprise one or more paratopes and/or antigen-binding sequences that are identical or similar to the anti-Ficolin-3 antibody FCN308, FCN334, and FCN329 paratopes and/or antigen-binding sequences described herein.

So, in a first broad aspect the present invention relates to methods and compounds suitable for inhibiting complement activation in a human body fluid by inhibition of Ficolin-3 (i.e. ficolin-3 inhibitor), such as by inhibiting the ability of ficolin-3 to associate with any of its natural ligand. Any suitable compound may be used to inhibit ficolin-3, such as any soluble natural ligand or other non-natural ligand or antagonist that do not activate complement. Alternatively, inhibitory antibodies against ficolin-3 may be used.

In a second aspect the present invention relates to an isolated recombinant human monoclonal antibody, or an antigen-binding fragment thereof, which exhibit specific binding to human Ficolin-3 and which inhibits complement activation in a human body fluid.

In a third aspect the present invention relates to a composition comprising an isolated recombinant human monoclonal antibody, or an antigen-binding fragment thereof or other ficolin-3 inhibitor, which exhibit specific binding to human Ficolin-3 and which inhibits complement activation in a human body fluid.

In a further aspect the present invention relates to an expression vector comprising a nucleotide sequence encoding an isolated recombinant human monoclonal antibody, or an antigen-binding fragment thereof, which exhibit specific binding to human Ficolin-3 and which inhibits complement activation in a human body fluid.

In a further aspect the present invention relates to a recombinant eukaryotic or prokaryotic host cell which produces an isolated recombinant human monoclonal antibody, or an antigen-binding fragment thereof, which exhibit specific binding to human Ficolin-3 and which inhibits complement activation in a human body fluid.

In a further aspect the present invention relates to a hybridoma which produces an isolated recombinant human monoclonal antibody, or an antigen-binding fragment thereof, which exhibit specific binding to human Ficolin-3 and which inhibits complement activation in a human body fluid.

In a further aspect the present invention relates to a method of producing an anti-Ficolin-3 antibody, or an antigen-binding fragment thereof according to the present invention, comprising culturing a host cell comprising a nucleic acid encoding said antibody under suitable conditions and recovering said antibody or antigen-binding fragment thereof.

In a further aspect the present invention relates to the use of an antibody or antigen-binding fragment of the invention or other ficolin-3 inhibitor for the inhibition of ficolin-3 recognition to its natural ligands.

In a further aspect the present invention relates to a method for treating an indication or condition associated with ficolin-3 ligand recognition, such as an indication associated with inflammation, coagulation, apoptosis and/or autoimmunity comprising administering the antibody or antigen-binding fragment or other ficolin-3 inhibitor according to the invention to a human subject suffering from or at risk for such an indication or condition.

In a further aspect the present invention relates to a method for inhibiting complement activation in a subject in need thereof the method comprising administering an antibody or antigen-binding fragment or other ficolin-3 inhibitor according to the invention to a human subject in need thereof.

In a further aspect the present invention relates to a pharmaceutical composition comprising an antibody or antigen-binding fragment or other ficolin-3 inhibitor according to the invention, and a pharmaceutically acceptable carrier.

In a further aspect the present invention relates to an antibody or other ficolin-3 inhibitor according to the invention for use as a medicament. In some embodiments the use is for treatment of an indication, condition or disease as defined herein.

In a further aspect the present invention relates to a diagnostic composition comprising an antibody or other ficolin-3 inhibitor as defined herein.

In a further aspect the present invention relates to a method for detecting the presence of human Ficolin-3 in a sample, the method comprising the steps of:
a) contacting the sample with an anti-Ficolin-3 antibody or other ficolin-3 inhibitor as defined herein under conditions that allow for formation of a complex between the antibody and human Ficolin-3; and
b) analyzing whether a complex has been formed.

In a further aspect the present invention relates to a kit for detecting the presence of human Ficolin-3 in a sample comprising
a) an anti-Ficolin-3 antibody or other ficolin-3 inhibitor as defined herein; and
b) instructions for use of the kit.

### LEGENDS TO THE FIGURE

FIG. 1: Panel of Ficolin-3 antibodies. rFicolin-3 was coated directly in a microtiter plate [1 μg/ml] ON 4° C. Supernatants containing mouse-anti-Ficolin-3 antibodies was diluted 1:10, added to the wells and incubated for 2 hours RT. Antibody binding was detected with rabbit-anti-mouse-HRP for 1 hour RT and the plate was developed using OPD substrate solution. Optical density was measured at 490 nm.

FIG. 2: Effect of antibodies on binding of recombinant Ficolin-3 to ligand (acBSA). As ligand for Ficolin-3 acBSA was coated to microtiter wells [5 μg/ml] in PBS ON at 4° C. rFicolin-3 [2 μg/ml] and the different antibodies in 2-fold dilution preincubated at 4° C. for 30 min. before added to the ELISA plate and incubated for 2 hours at 37° C. Detection of Ficolin-3 binding was performed with biotinylated mAb mouse FCN334 and subsequently strep-HRP. Between all steps plates were washed with barbital buffer containing 0.05% Tween 20. Graph shows antibody dosage effect on Ficolin-3 binding: FCN304 (-|-), FCN308 (--), FCN319 (-*-), FCN329 (-x-), FCN334 (-▴-), nonspecific isotype control Ciona IgG (-▪-), control with no antibody (--).

FIG. 3: Effect of antibodies on binding of serum Ficolin-3 to ligand (acBSA). AcBSA was coated on to microtiter wells [5 μg/ml] in PBS ON 4° C. Serum [1:50] and the different antibodies in 2-fold dilution preincubated at 4° C. for 30 min. before added to the ELISA plate and incubated for 2 hours at 37° C. Detection of Ficolin-3 binding was performed with biotinylated FCN334 and subsequently strep-HRP. Between all steps plates were washed with barbital buffer containing 0.05% Tween 20. Graph shows antibody dosage effect on Ficolin-3 binding: FCN304 (-|-), FCN308 (--), FCN319 (-*-), FCN329 (-x-), FCN334 (-▴-), nonspecific isotype control Ciona IgG (-▪-), control with no antibody (--).

FIG. 4: Effect of antibodies on deposition of complement factor C4. AcBSA was coated on to microtiter wells [5 μg/ml] in PBS ON 4° C. First, full serum preincubated with SPS [0.5 mg/ml] for 5 minutes on ice. Second, serum [1:80] preincubated with antibody 30 minutes at 4° C. and was then added to the ELISA plate for 30 minutes at 37° C. C4-deposition on acBSA was detected with pAb rabbit-anti-huC4 (from DAKO) and subsequently donkey-anti-rabbit-HRP. Between all steps plates were washed with barbital buffer containing 0.05% Tween 20.

FIG. 5: Effect of antibodies on deposition of complement factor C3. AcBSA was coated on to microtiter wells [5 μg/ml] in PBS ON 4° C. First, full serum preincubated with SPS [0.5 mg/ml] for 5 minutes on ice. Second, serum [1:80] preincubated with antibody 30 minutes at 4° C. and was then added to the ELISA plate for 30 minutes at 37° C. C3-deposition on acBSA was detected with pAb rabbit-anti-huC3 (from Dade Behring) and subsequently donkey-anti-rabbit-HRP. Between all steps plates were washed with barbital buffer containing 0.05% Tween 20.

FIG. 6: Effect of antibodies on deposition of terminal complement complex (TCC). AcBSA was coated on to microtiter wells [5 μg/ml] in PBS ON 4° C. First, full serum preincubated with SPS [0.5 mg/ml] for 5 minutes on ice. Second, serum [1:20] preincubated with antibody 30 minutes at 4° C. and was then added to the ELISA plate for 45 minutes at 37° C. TCC-formation on acBSA was detected with biotinylated mouse-anti-C5b-9 (from Bioporto Diagnostic) and subsequently strep-HRP. Between all steps plates were washed with barbital buffer containing 0.05% Tween 20.

FIG. 7: Kaplan-Meier survival plot of 527 patients followed for one year kidney graft survival. The grey line indicates high ficolin-3 levels (>0.46 microgram/ml) while the black line Indicates low ficolin-3 levels (<0.46 microgram/ml).

FIG. 8: Inhibition of rFcolin-3 binding to necrotic cells with FCN308. The plots show FACS densities of Ficolin-3 binding to necrotic cells in the presence of varying amounts of antibody FCN308.

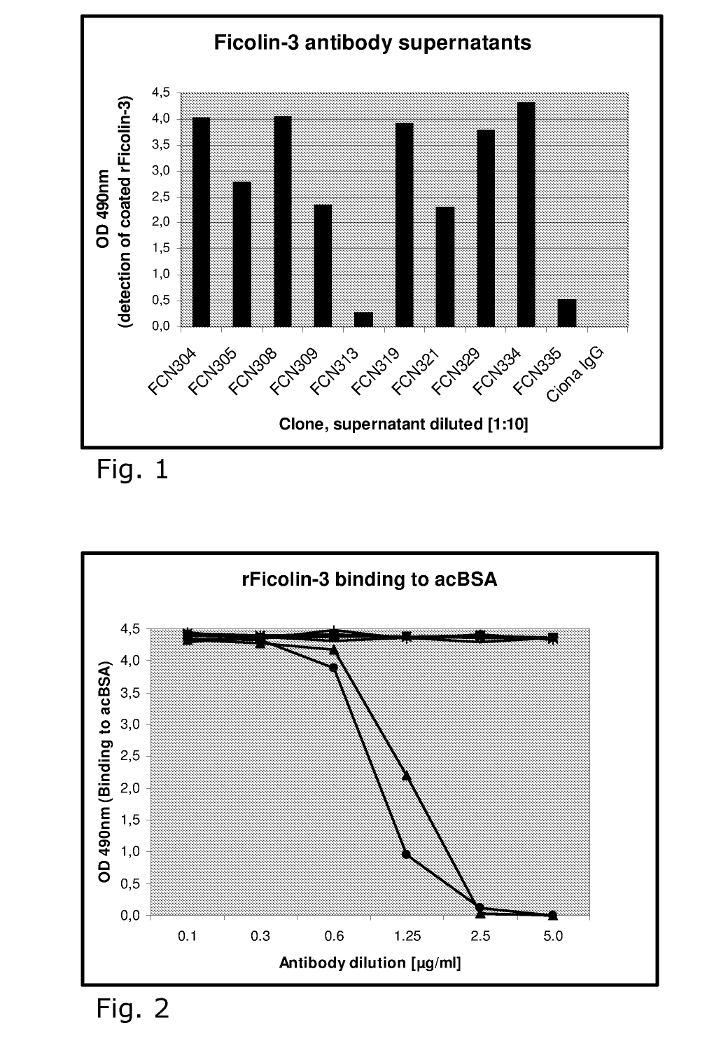

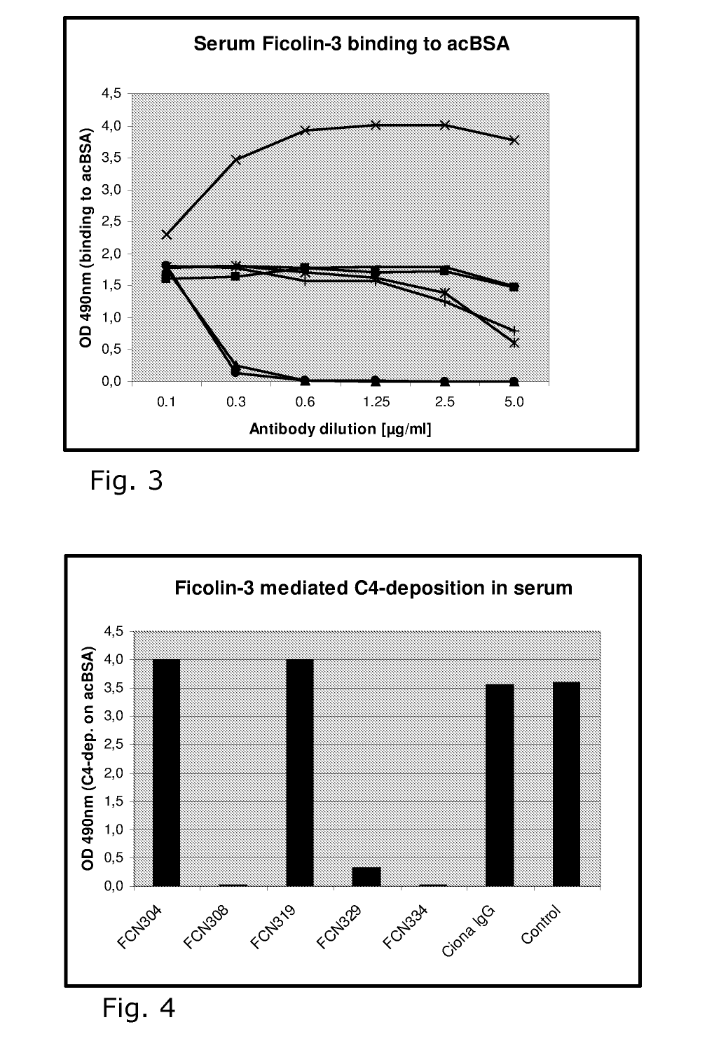

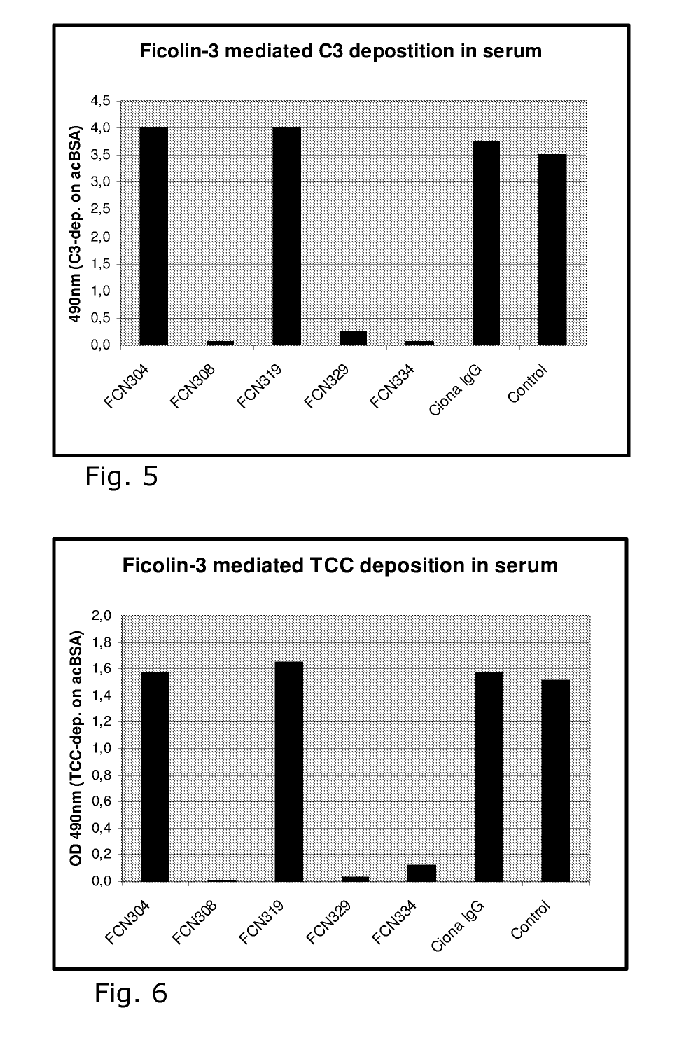

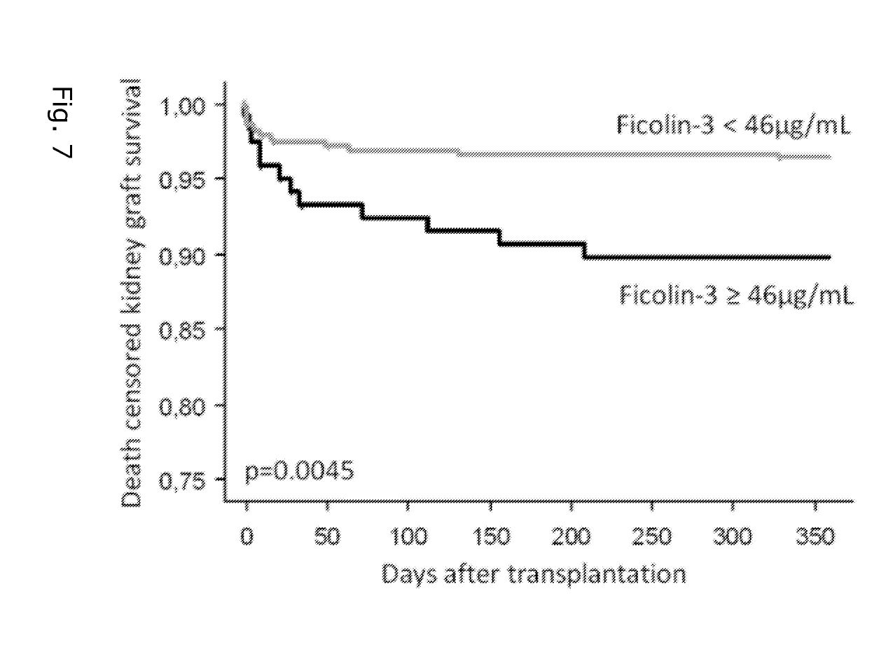

### DETAILED DISCLOSURE OF THE INVENTION

The inventors of the present invention have discovered that it is possible to inhibit complement activation by inhibition of ficolin-3 binding to ligands. This has been exemplified by using monoclonal antibodies towards ficolin-3.

The ficolin-3 gene is pseudogene in different species used for pre-clinical animal testing and no good animal models exist for testing ficolin-3 mediated effect. This has been circumvented by generating a panel of mouse monoclonal antibodies against human ficolin-3 in order to achieve inhibition of ficolin-3 binding to targets and subsequent activation of downstream effector functions. The inventors of the present invention have obtained antibodies that completely block binding of ficolin-3 to acetylated albumin and to dying host cells and which completely block downstream complement deposition.

The inventors of the present invention have thus discovered that certain antibodies against human ficolin-3 may be used for inhibition of ficolin-3 recognition to its ligands. Thereby these antibodies may inhibit downstream ficolin-3 mediated functions, including the inhibition of the complement pathway of the immune system. Accordingly antibodies according to the present invention may be used in the treatment of specific medical conditions associated herewith including inhibition of adverse effects associated with inflammation and coagulation.

Uncontrolled activation of the complement system and/or the coagulation cascade is strongly associated with fatal severe outcome in variety of diseases ranging from systemic inflammation and sepsis, through myocardial infarction and autoimmunity. Accordingly functional inhibitors, such as the antibodies against human ficolin-3 according to the present invention may be very useful for the control of the complement system and the coagulation cascade.

Inhibition of coagulation and complement activation has been shown to be a promising therapeutic tool.

This present invention describes both a possible novel use of antibodies against ficolin-3 as an inhibitor of complement and coagulation functions. However, the antibodies according to the present invention may also be used for other functions, such as a scavenger and/or a signalling function or an inhibitor of such function. Moreover, it may be used in bioassays, including the quantitative measurement of human ficolin-3, or immunohistochemical detection of human ficolin-3 in different tissues such as in the diagnostic disease settings, including malignant diseases, autoimmune, metabolic and/or inflammatory conditions.

### DEFINITIONS

As used herein, “human Ficolin-3”, refer to a human ficolin-3 isoform 1 with UniProtKB/Swiss-Prot identifier 075636-1 (FCN3_HUMAN), NCBI Reference Sequence: NP_{—}003656.2, (SEQ ID NO:1) with a mRNA of SEQ ID NO:3, or naturally occurring variants and isoforms thereof. In one embodiment human Ficolin-3 refers to isoform 1 with SEQ ID NO:1.

The term “complement activity” as used herein means the ability activate the complement system. The complement activity may be measured with assay as described in the section headed “Assays”.

As used herein “inhibits complement activity” refers to any in vitro measurable decrease in deposition of terminal complement complex (TCC) or upstream ficolin-3 complement dependent deposition and activation. The inhibition of complement activity may be measured in the assays described herein or in any other assay known to the person skilled in the art.

In the present context, the term “treatment” is meant to include both prevention of an expected condition involving inappropriate complement activation, such as inflammation and reperfusion injury and regulation of an already occurring condition, such as myocardial Infarction and stroke with the purpose of inhibiting or minimising the tissue damage Prophylactic administration of the anti-ficolin-3 antibodies according to the present invention is thus included in the term “treatment”.

The term “subject” as used herein is intended to mean any animal, in particular mammals, such as humans, and may, where appropriate, be used interchangeably with the term “patient”.

The term “antibody” herein is used in the broadest sense and specifically includes full-length monoclonal antibodies, polyclonal antibodies, and, unless otherwise stated or contradicted by context, antigen-binding fragments, antibody variants, and multispecific molecules thereof, so long as they exhibit the desired biological activity. Generally, a full-length antibody is a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarily determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. General principles of antibody molecule structure and various techniques relevant to the production of antibodies are provided in, e.g., Harlow and Lane, ANTIBODIES: A LABORATORY MANUAL, Cold Spring Harbor Laboratory Press, Cold Spring Harbor, N.Y., (1988).

An “antigen-binding fragment” of an antibody is a molecule that comprises a portion of a full-length antibody which is capable of detectably binding to the antigen, typically comprising one or more portions of at least the VH region. Antigen-binding fragments include multivalent molecules comprising one, two, three, or more antigen-binding portions of an antibody, and single-chain constructs wherein the VL and VH regions, or selected portions thereof, are joined by synthetic linkers or by recombinant methods to form a functional, antigen-binding molecule. While some antigen-binding fragments of an antibody can be obtained by actual fragmentation of a larger antibody molecule (e.g., enzymatic cleavage), most are typically produced by recombinant techniques. The terms “antibody derivative” and “immunoconjugate” are used interchangeably herein to denote molecules comprising a full-length antibody or an antigen-binding fragment thereof, wherein one or more amino acids are chemically modified, e.g., by alkylation, PEGylation, acylation, ester formation or amide formation or the like, e.g., for linking the antibody to a second molecule. Exemplary modifications include PEGylation (e.g., cysteine-PEGylation), biotinylation, radiolabelling, and conjugation with a second agent (such as a cytotoxic agent),

A “multispecific molecule” comprises an antibody, or an antigen-binding fragment thereof, which is associated with or linked to at least one other functional molecule (e.g. another peptide or protein such as another antibody or ligand for a receptor) thereby forming a molecule that binds to at least two different binding sites or target molecules. Exemplary multispecific molecules include bi-specific antibodies and antibodies linked to soluble receptor fragments or ligands.

The term “human antibody”, as used herein, is intended to include antibodies having variable regions in which both the framework and CDR regions are derived from (i.e., are identical or essentially identical to) human germline immunoglobulin sequences. Furthermore, if the antibody contains a constant region, the constant region also is “derived from” human germline immunoglobulin sequences. The human antibodies of the invention may include amino acid residues not encoded by human germline immunoglobulin sequences (e.g., mutations introduced by random or site-specific mutagenesis in vitro or by somatic mutation in viva). However, the term “human antibody”, as used herein, is not intended to include antibodies in which CDR sequences derived from the germline of another species, such as a mouse or any other species, have been grafted onto human framework sequences.

A “humanized” antibody is a human/non-human chimeric antibody that contains a minimal sequence derived from non-human immunoglobulin. For the most part, humanized antibodies are human immunoglobulins (recipient antibody) in which residues from a hyper-variable region of the recipient are replaced by residues from a hypervariable region of a non-human species (donor antibody) such as mouse, rat, rabbit, or non-human primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, a humanized antibody will comprise substantially all of at least one, and typically two, variable domains, in which all or substantially all of the hypervariable loops correspond to those of a non-human immunoglobulin and all or substantially all of the FR residues are those of a human immunoglobulin sequence. The humanized antibody can optionally also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992), WO 92/02190, US 2006/0073137, U.S. Pat. No. 6,750,325, U.S. Pat. No. 6,632,927, U.S. Pat. No. 6,639,055, U.S. Pat. No. 6,548,640, U.S. Pat. No. 6,407,213, U.S. Pat. No. 6,180,370, U.S. Pat. No. 6,054,297, U.S. Pat. No. 5,929,212, U.S. Pat. No. 5,895,205, U.S. Pat. No. 5,886,152, U.S. Pat. No. 5,877,293, U.S. Pat. No. 5,869,619, U.S. Pat. No. 5,821,337, U.S. Pat. No. 5,821,123, U.S. Pat. No. 5,770,196, U.S. Pat. No. 5,777,085, U.S. Pat. No. 5,766,886, U.S. Pat. No. 5,714,350, U.S. Pat. No. 5,693,762, U.S. Pat. No. 5,693,761, U.S. Pat. No. 5,530,101, U.S. Pat. No. 5,585,089, and U.S. Pat. No. 5,225,539.

The term “hypervariable region” when used herein refers to the amino acid residues of an antibody that are responsible for antigen binding. The hypervariable region generally comprises amino acid residues from a “complementarity-determining region” or “CDR” (residues 24-34 (L1), 50-56 (L2) and 89-97 (L3) in the light-chain variable domain and 31-35 (H1), 50-65 (H2) and 95-102 (H3) in the heavy-chain variable domain; (Kabat et al. (1991) Sequences of Proteins of Immunological Interest, Fifth Edition, U.S. Department of Health and Human Services, NIH Publication No. 91-3242) and/or those residues from a “hypervariable loop” (residues 26-32 (L1), 50-52 (L2) and 91-96 (L3) in the light-chain variable domain and 26-32 (H1), 53-55 (H2) and 96-101 (H3) in the heavy-chain variable domain; Chothia and Lesk, J. Mol. Biol 1987; 196:901-917). Typically, the numbering of amino acid residues in this region is performed by the method described in Kabat et al., supra. Phrases such as “Kabat position”, “variable domain residue numbering as in Kabat” and “according to Kabat” herein refer to this numbering system for heavy chain variable domains or light chain variable domains. Using the Kabat numbering system, the actual linear amino acid sequence of a peptide may contain fewer or additional amino acids corresponding to a shortening of, or insertion into, a FR or CDR of the variable domain. For example, a heavy chain variable domain may include a single amino acid insert (residue 52a according to Kabat) after residue 52 of CDR H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

“Framework region” or “FR” residues are those VH or VL residues other than the CDRs as herein defined.

An “epitope” or “binding site” is an area or region on an antigen to which an antigen-binding peptide (such as an antibody) specifically binds. A protein epitope may comprise amino acid residues directly involved in the binding (also called the immunodominant component of the epitope) and other amino acid residues, which are not directly involved in the binding, such as amino acid residues which are effectively blocked by the specifically antigen binding peptide (in other words, the amino acid residue is within the “solvent-excluded surface” and/or “footprint” of the specifically antigen binding peptide). The term epitope herein includes both types of amino acid binding sites in any particular region of a human Ficolin-3 that specifically binds to an anti-human Ficolin-3 antibody, or another human Ficolin-3-specific agent according to the invention, unless otherwise stated (e.g., in some contexts the Invention relates to antibodies that bind directly to particular amino acid residues). Ficolin-3 may comprise a number of different epitopes, which may include, without limitation, (1) linear peptide antigenic determinants, (2) conformational antigenic determinants which consist of one or more noncontiguous amino acids located near each other in a mature Ficolin-3 conformation; and (3) post-translational antigenic determinants which consist, either in whole or part, of molecular structures covalently attached to a Ficolin-3, such as carbohydrate groups. Unless otherwise specified or contradicted by context, conformational antigenic determinants comprise Ficolin-3 amino acid residues within an about 4 Å distance from an atom of an antigen-binding peptide.

The “solvent excluded surface” is the area of a molecule which, in a computer calculation, cannot be reached by any water molecule, e.g., because of binding of the molecule to a ligand (Lee and Richards, J Mol Biol 1971; 55:379-400, which is incorporated herein by reference).

The phrase “binds to essentially the same epitope or determinant as” an antibody of interest means that an antibody “competes” with the antibody of interest for Ficolin-3 molecules to which the antibody of interest specifically binds.

A “paratope” is an area or region of an antigen-binding portion of an antibody that specifically binds an antigen. Unless otherwise stated or clearly contradicted by context, a paratope may comprise amino acid residues directly involved in epitope binding, several of which are typically in CDRs, and other amino acid residues, which are not directly involved in the binding, such as amino acid residues which are effectively blocked by the specifically bound antigen (in other words, the amino acid residue is within the “solvent-excluded surface” and/or “footprint” of the specifically bound antigen).

The ability of an anti-Ficolin-3 antibody to “block” the binding of a Ficolin-3 molecule to a natural Ficolin-3-ligand, means that the antibody, in an assay using soluble or cell-surface associated Ficolin-3 and ligand molecules, can detectably reduce the binding of a Ficolin-3-molecule to the ligand in a dose-dependent fashion, where the Ficolin-3 molecule detectably binds to the ligand in the absence of the antibody.

A “variant” of a polypeptide refers to a polypeptide having an amino acid sequence that is substantially identical to a reference polypeptide, typically a native or “parent” polypeptide. The polypeptide variant may possess one or more amino acid substitutions, deletions, and/or Insertions at certain positions within the native amino acid sequence and/or additions at one or both termini.

The term “substantially identical” in the context of two amino acid sequences means that the sequences, when optimally aligned, such as by the programs GAP or BEST-FIT using default gap weights, share at least about 50 percent sequence identity. Typically sequences that are substantially identical will exhibit at least about 60, at least about 70, at least about 80, at least about 90, at least about 95, at least about 98, or at least about 99 percent sequence identity.

“Corresponding” amino acid positions in two substantially identical amino acid sequences are those aligned by any of the protein analysis software referred to herein.

A nucleic acid sequence (or element) is “operably linked” to another nucleic acid sequence (or element) when it is placed into a functional relationship with the other nucleic acid sequence. For example, DNA for a pre-sequence or secretory leader is operably linked to DNA for (i.e. coding for expression of) a polypeptide if it is expressed as a pre-protein that participates in the secretion of the polypeptide; a promoter or enhancer is operably linked to a coding sequence if it affects the transcription of the sequence; or a ribosome-binding site is operably linked to a coding sequence if it is positioned so as to facilitate translation. Generally, “operably linked” means that the DNA sequences being linked are contiguous, and, in the case of a secretory leader, contiguous and in reading phase. However, some elements, such as enhancers, do not have to be contiguous with a coding sequence in order to be operably linked. Linking typically is accomplished by ligation at convenient restriction sites. If such sites do not exist, the synthetic oligonucleotide adaptors or linkers may be used in accordance with conventional practice.

An “isolated” molecule is a molecule that is the predominant species in the composition wherein it is found with respect to the class of molecules to which it belongs (i.e., it makes up at least about 50% of the type of molecule in the composition and typically will make up at least about 70%, at least about 80%, at least about 85%, at least about 90%, at least about 95%, or more of the species of molecule, e.g., peptide, in the composition). Commonly, a composition of an antibody molecule will exhibit 98%, 98%, or 99% homogeneity for antibody molecules in the context of all present peptide species in the composition or at least with respect to substantially active peptide species in the context of proposed use.

In the context of the present invention, “treatment” or “treating” refers to preventing, alleviating, managing, curing or reducing one or more symptoms or clinically relevant manifestations of a disease or disorder, unless contradicted by context. For example, “treatment” of a patient in whom no symptoms or clinically relevant manifestations of a disease or disorder have been identified is preventive or prophylactic therapy, whereas clinical, curative, or palliative “treatment” of a patient in whom symptoms or clinically relevant manifestations of a disease or disorder have been identified generally does not constitute preventive or prophylactic therapy. Each form of treatment may be considered a distinct aspect of the invention.

The present invention is based, in part, on anti-Ficolin-3 antibodies with properties suitable for treating human patients that require an inhibition of the complement system. Antibodies of the invention are typically either fully human or humanized in order to minimize the risk for an immune response against the antibody by the patient's own immune system, and bind to human Ficolin-3.

In one aspect, the present invention provides a fully human antibody, or antigen-binding fragment thereof, that effectively prevents Ficolin-3-mediated complement activation; has an affinity to human Ficolin-3 of 10 nM or less, and is non-depleting, e.g., by having an IgG4 isotype. In a particular embodiment, the antibody is a non-depleting fully human antibody of the IgG4 isotype, with an affinity to human Ficolin-3 of 1 nM or less, preferably 300 μM or less, which blocks at least 50%, at least 70%, or at least 90% of endogenous human Ficolin-3-ligand binding. In another particular embodiment, the antibody is a bivalent non-depleting fully human antibody of the IgG4 isotype, with an affinity below 100 μM.

The production, characterization, and use of antibodies specifically binding human Ficolin-3 and having some or all of these properties are described in more detail in the following sections, including the Examples.

### Anti-Ficolin-3 Antibodies

The antibodies of the invention are characterized by particular functional and/or structural features or properties. Assays to evaluate the functional activities of anti-human Ficolin-3 antibodies are described in detail in the Examples, and structural properties such as, e.g., amino acid sequences, are described below. Functional properties

The antibodies of the invention bind to human Ficolin-3. In one embodiment, an antibody of the invention binds to human Ficolin-3 with high affinity, for example with a KD of 10⁻⁷ M or less, a KD of 10⁻⁸ M or less, a KD of 1 nM or less, a KD of 0.3 nM or less, a KD of 0.2 nM or less, 0.1 nM or less, 0.05 nM or less, or 0.01 nM or less. In a particular embodiment, the antibody binds to human Ficolin-3 with an affinity of 0.1 nM or less.

In one aspect, the invention provides antibodies also binding to one or more Ficolin-3 orthologs in monkey such as cynomolgous monkey. Additionally or alternatively, an antibody can bind to cynomolgous or rhesus Ficolin-3 with an affinity of about 30% or more, about 50% or more, about 65% or more, or about 75% or more, about 80% or more, about 85% or more, or about 90% or more, of the affinity for human Ficolin-3. Such antibodies have the advantage of allowing for toxicity testing in the most suitable animal model (or models) prior to use in humans.

In one particular aspect, antibodies of the invention also bind a form of Ficolin-3 that known murine anti-human Ficolin-3 antibodies.

In another embodiment, the invention provides antibodies that compete with and/or bind to the same epitope on human Ficolin-3 as anti-Ficolin-3 antibody FCN308, FCN334, and FCN329 described herein. Such antibodies can be identified based on their ability to cross-compete with anti-Ficolin-3 antibody FCN308, FCN334, and FCN329 in standard human Ficolin-3 binding assays as described herein. The ability of a test antibody to inhibit the binding of FCN308, FCN334, and FCN329 to human Ficolin-3 demonstrates that the test antibody can compete with FCN308, FCN334, and FCN329 for binding to human Ficolin-3 and thus can bind to the same epitope on human Ficolin-3 as antibody FCN308, FCN334, and FCN329 described herein. In a preferred embodiment, the antibody that binds to the same epitope on human Ficolin-3 as antibody FCN308, FCN334, and FCN329 is a human monoclonal antibody. Such human monoclonal antibodies can be prepared and isolated as described in the Examples described herein.

As used herein, a human antibody comprises heavy or light chain variable regions “of” or “derived from” or that are “the product of” a particular germline sequence if the variable regions of the antibody are obtained from a system (as described below) that uses human germline immunoglobulin genes. Such “systems” include immunizing a transgenic mouse carrying human immunoglobulin genes with the antigen of interest or screening a human immunoglobulin gene library displayed on phage with the antigen of interest. A human antibody that is “of” or “derived from” or “the product of” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody. A human antibody that is “of” or “derived from” or “the product of” a particular human germline immunoglobulin sequence may contain amino acid differences as compared to the germline sequence, due to, for example, naturally-occurring somatic mutations or intentional introduction of site-directed mutation(s) (which may be selected substitutions).

However, a human antibody is typically at least 90% identical in amino acid sequence to an amino acid sequence encoded by a recombined germline immunoglobulin sequence and can usually be identified as human when compared to the germline immunoglobulin amino acid sequences of other species (e.g., murine germline sequences). In certain cases, a human antibody may be at least 95%, or even at least 96%, 97%, 98%, or 99% identical in amino acid sequence to the amino acid sequence encoded by the recombined germline immunoglobulin gene.

Typically, a human antibody derived from a particular human germline sequence will display no more than 10 amino acid differences from the amino acid sequence encoded by the human germline immunoglobulin gene. In certain cases, the human antibody may display no more than 8, no more than 5, or even no more than 4, 3, 2, or 1 amino acid difference, or no amino acid difference, from the amino acid sequence encoded by the recombined germline immunoglobulin gene.

In yet another embodiment, an antibody of the invention comprises heavy and light chain variable regions comprising amino acid sequences that are homologous to the amino acid sequences of the preferred antibodies described herein, and wherein the antibodies retain the desired functional properties of the anti-human Ficolin-3 antibodies of the invention. For example, the invention provides an isolated antibody, or antigen binding portion thereof, comprising a heavy chain variable region and a light chain variable region, wherein: (a) the VH region comprises an amino acid sequence that is at least 80% identical to a reference antibody sequence; (b) the VL region comprises an amino acid sequence that is at least 80% identical to a reference antibody sequence; (c) the antibody binds to human Ficolin-3 and exhibits at least one of the functional properties described herein, preferably several of the functional properties described herein.

In other embodiments, the VH and/or VL amino acid sequences may be 85%, 90%, 95%, 96%, 97%, 98% or 99% identical to the reference antibody sequence. An antibody having VH and VL regions having high (i.e. 80% or greater) identity to the VH and VL regions of the sequences set forth above, can be obtained by mutagenesis (e.g., site-directed or PCR-mediated mutagenesis) of nucleic acid molecules encoding a reference antibody sequence, followed by testing of the encoded altered antibody for retained function (e.g., human Ficolin-3 binding affinity, human Ficolin-3-ligand blocking.

The percent identity between the two sequences is a function of the number of identical positions shared by the sequences (i.e., % identity=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm in sequence-analysis software. Protein analysis software matches similar sequences using measures of similarity assigned to various substitutions, deletions and other modifications, including conservative amino acid substitutions.

The percent identity between two amino acid sequences can be determined, e.g., using the Needleman and Wunsch (J. Mol. Biol. 48:444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. Polypeptide sequences can also be compared using FASTA, applying default or recommended parameters. A program in GCG Version 6.1., FASTA (e.g., FASTA2 and FASTA3) provides alignments and percent sequence identity of the regions of the best over-lap between the query and search sequences (Pearson, Methods Enzymol. 1990; 183:63-98; Pearson, Methods Mol. Biol. 2000; 132: 185-219). The percent identity between two amino acid sequences can also be determined using the algorithm of E. Meyers and W. Miller (Comput. Appl. Biosci., 1988; 11-17) which has been incorporated into the ALIGN program (version 2.0), using a PAM120 weight residue table, a gap length penalty of 12 and a gap penalty of 4. Another algorithm for comparing a sequence to any other sequence contained in a database is the computer program BLAST, especially blastp, using default parameters. See, e.g., Altschul et al., J. Mol. Biol. 1990; 215:403-410; Altschul et al., Nucleic Acids Res. 1997; 25:3389-402 (1997); each herein incorporated by reference. The protein sequences of the present invention can there be used as a “query sequence” to perform a search against public databases to, for example, identify related sequences. Such searches can be performed using the XBLAST program (version 2.0) of Altschul, et al. 1990 (supra). BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al., 1997 (supra). When utilizing BLAST and Gapped BLAST programs, the default parameters of the respective programs (e.g., XBLAST and NBLAST) can be used. See http://www.ncbi.nlm.nih.gov.

In certain embodiments, an antibody of the invention comprises a VH region comprising CDR1, CDR2 and CDR3 sequences and a VL region comprising CDR1, CDR2 and CDR3 sequences, wherein one or more of these CDR sequences comprise specified amino acid sequences based on the preferred reference antibodies described herein, wherein one or more CDRs optionally contains one or more conservative amino acid modifications, and wherein the antibodies retain the desired functional properties of the anti-human Ficolin-3 antibodies of the invention. Accordingly, the invention provides an isolated antibody, or antigen-binding fragment thereof, comprising a heavy chain variable region comprising CDR1, CDR2, and CDR3 sequences and a light chain variable region comprising CDR1, CDR2, and CDR3 sequences, wherein: (a) the VH region CDR3 sequence comprises an amino acid sequence of the reference antibody sequence; (b) the VL region CDR3 sequence comprises an amino acid sequence of the reference antibody sequence; (c) one or more CDRs optionally contains one or more conservative amino acid modifications, and (d) the antibody binds to human Ficolin-3 and exhibits at least one of the functional properties described herein.

In a further embodiment, the VH region CDR2 sequence comprises an amino acid sequence selected of the reference antibody sequence; and the VL region CDR2 sequence comprises an amino acid sequence of the reference antibody sequence, wherein one or more CDRs optionally contains one or more conservative amino acid modifications.

In a still further embodiment, the VH region CDR1 sequence comprises an amino acid sequence of the reference antibody sequence, and conservative modifications thereof; and the VL region CDR1 sequence comprises an amino acid sequence of the reference antibody sequence, wherein one or more CDRs optionally contains one or more conservative amino acid modifications.

As used herein, the term “conservative amino acid modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as, e.g., site-directed mutagenesis and PCR-mediated mutagenesis. An antibody sequence comprising amino acid modifications as compared to a parent antibody is typically at least 90%, preferably at least 95%, 98%, or 99% identical to the corresponding amino acid sequence in the parent and/or comprises at most 10, preferably at most 5, 4, 3, 2 amino acid modifications as compared to the parent antibody sequence.

“Conservative” amino acid substitutions are typically those in which an amino acid residue is replaced with an amino acid residue having a side chain with similar physico-chemical properties. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g. glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g. threonine, valine, isoleucine) and aromatic side chains (e.g., tyrosine, phenylalanine, tryptophan, histidine).

Thus, one or more amino acid residues within the CDR regions of an antibody of the invention can be replaced with other amino acid residues from the same side chain family and the altered antibody can be tested for retained function described herein.

The anti-human Ficolin-3 antibodies of the invention may be prepared as full-length antibodies or antigen-binding fragments thereof. Examples of antigen-binding fragments include Fab, Fab′, F(ab)2, F(ab′)2, F(ab)3, Fv (typically the VL and VH domains of a single arm of an antibody), single-chain Fv (scFv; see e.g., Bird et al., Science 1988; 242:423-426; and Huston et al. PNAS 1988; 85:5879-5883), dsFv, Fd (typically the VH and CH 1 domain), and dAb (typically a VH domain) fragments; VH, VL, VhH, and V-NAR domains; monovalent molecules comprising a single VH and a single VL chain; minibodies, diabodies, triabodies, tetrabodies, and kappa bodies (see, e.g., Ill et al., Protein Eng 1997; 10:949-57); camel IgG; IgNAR; as well as one or more isolated CDRs or a functional paratope, where the isolated CDRs or antigen-binding residues or polypeptides can be associated or linked together so as to form a functional antibody fragment. Various types of antibody fragments have been described or reviewed in, e.g., Holliger and Hudson, Nat Biotechnol 2005; 23:1126-1136; WO 2005/040219, US 2005/0238646 and US 2002/0161201. Antibody fragments can be obtained using conventional recombinant or protein engineering techniques, and the fragments can be screened for antigen-binding or other function in the same manner as are intact antibodies.

Various techniques have been developed for the production of antibody fragments. Traditionally, these fragments were derived via proteolytic digestion of full-length antibodies (see, e.g., Morimoto et al., Journal of Biochemical and Biophysical Methods, 24:107-117 (1992); and Brennan et al, Science, 229:81 (1985)). However, these fragments can now be produced directly by recombinant host cells. Alternatively, Fab′-SH fragments can be directly recovered from E. coli and chemically coupled to form F(ab′)2 fragments (Carter et al., Bio/Technology, 10:163-167 (1992)). According to another approach, F(ab′)2 fragments can be isolated directly from recombinant host cell culture. In other embodiments, the antibody of choice is a single-chain Fv fragment (scFv). See WO 1993/16185; U.S. Pat. No. 5,571,894; and U.S. Pat. No. 5,587,458. The antibody fragment may also be a “linear antibody”, e.g., as described in U.S. Pat. No. 5,641,870, for example. Such linear antibody fragments may be monospecific or bispecific.

In another aspect, the present invention features multispecific molecules comprising an anti-human Ficolin-3 antibody, or an antigen-fragment thereof, of the invention. Such multispecific molecules include bispecific molecules comprising at least one first binding specificity for human Ficolin-3 and a second binding specificity for a second target epitope. One type of bispecific molecules are bispecific antibodies. Bispecific antibodies are antibodies that have binding specificities for at least two different epitopes. Methods for making bispecific antibodies are known in the art, and traditional production of full-length bispecific antibodies is usually based on the coexpression of two immunoglobulin heavy-chain-light-chain pairs, where the two chains have different specificities (Millstein et al., Nature, 305: 537-539 (1983)). Bispecific antibodies can be prepared as full-length antibodies or antibody fragments (e.g. F(ab′)2 bispeciflc antibodies) or any other antigen-binding fragments described herein.

In the bispecific antibodies according to the present invention, at least one binding epitope is on the human Ficolin-3 protein. The anti-Ficolin-3-binding moiety may be combined with second moiety that binds to any other molecule, so as to inhibit complement activation.

Other multispecific molecules include those produced from the fusion of a human Ficolin-3-binding antibody moiety to one or more other non-antibody proteins. Such multispecific proteins and how to construct them have been described in the art. See, e.g., Dreier et al. (Bioconjug. Chem. 9(4): 482-489 (1998)); U.S. Pat. No. 6,046,310; US 2003/0103984; EP 1413316; US 2004/0038339; von Strandmann et al., Blood (2006; 107: 1955-1962), and WO 2004/056873.

Multispecific molecules with more than two valencies are also contemplated. For example, trispecific antibodies can be prepared. Tutt et al., J. Immunol, 147: 60 (1991). The multispecific molecules of the present invention can be prepared by conjugating the constituent binding specificities using methods known in the art. For example, each binding specificity of the multispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-5-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl)cyclohaxane-1-carboxylate (sulfo-SMCC) (see e.g., Karpovsky et al. (1984) J. Exp. Med. 160:1686; Liu, M A et al. (1985) Proc. Natl. Acad. Sci. USA 82:8648). Other methods include those described in Paulus (1985) Behring Ins. Mitt. No. 78, 118-132; Brennan et al. (1985) Science 229:81-83), and Glennie et al. (1987) J. Immunol. 139: 2367-2375). Preferred conjugating agents are SATA and sulfo-SMCC, both available from Pierce Chemical Co. (Rockford, Ill.). When the binding specificities are antibodies, they can be conjugated via sulthydryl bonding of the C-terminus hinge regions of the two heavy chains. In a particularly preferred embodiment, the hinge region is modified to contain an odd number of sulfhydryl residues, preferably one, prior to conjugation.

Alternatively, both binding specificities can be encoded in the same vector and expressed and assembled in the same host cell. This method is particularly useful where the bispecific molecule is a mAb×mAb, mAb×Fab, Fab×F(ab′)2 or ligand×Fab fusion protein. A bispecific molecule of the invention can be a single chain molecule comprising one single chain antibody and a binding determinant, or a single chain bispecific molecule comprising two binding determinants. Bispecific molecules may comprise at least two single chain molecules. Methods for preparing bispecific molecules are described or reviewed in, for example in U.S. Pat. No. 5,260,203; U.S. Pat. No. 5,455,030; U.S. Pat. No. 4,881,175; U.S. Pat. No. 5,132,405; U.S. Pat. No. 5,091,513; U.S. Pat. No. 5,476,786; U.S. Pat. No. 5,013,653; U.S. Pat. No. 5,258,498; U.S. Pat. No. 5,482,858; US 2003/0078385, Kontermann et al., (2005) Acta Pharmacological Sinica 26(1): 1-9; Kostelny et al., (1992) J. Immunol. 148(5): 1547-1553; Hollinger et al., (1993) PNAS (USA) 90:6444-6448; and Gruber et al. (1994) J. Immunol. 152: 5368.

### Antibody Variants

An antibody of the invention further can be prepared using an antibody having one or more of the VH and/or VL sequences disclosed herein as starting material to engineer a modified antibody or antibody “variant”, which modified antibody may have altered properties from the parent antibody. An antibody can be engineered by modifying one or more residues within one or both variable regions (i.e. VH and/or VL), for example within one or more CDR regions and/or within one or more framework regions. Additionally or alternatively, an antibody can be engineered by modifying residues within the constant region(s), for example to alter the effector function(s) of the antibody. Additionally, from antigen-binding portions of an antibody, other constructs such as antigen-binding fragments, antibody derivatives, immuno-conjugates, and multispecific molecules can be prepared.

Standard molecular biology techniques can be used to prepare and express the altered antibody sequence.

Though an antibody variant or derivative typically has at least one altered property as compared to the “parent” antibody, the antibody variant or derivative can retain one, some or most of the functional properties of the anti-human Ficolin-3 antibodies described herein.

The functional properties of the antibody variants and derivatives can be assessed using standard assays available in the art and/or described herein. For example, the ability of the antibody to bind human Ficolin-3 can be determined using standard binding assays, such as those set forth in the Examples (e.g., Biacore, flow cytometry, or ELISAs).

### Variable Region Modifications

One type of variable region engineering that can be performed is CDR grafting. Antibodies interact with target antigens predominantly through amino acid residues that are located in the six heavy and light chain complementarily determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann, L. et al. (1998) Nature 332:323-327; Jones, P. et al. (1986) Nature 321:522-525; Queen, C. et al. (1989) Proc. Natl. Acad. Sci. U.S.A. 86:10029-10033; U.S. Pat. No. 5,225,539; U.S. Pat. No. 5,530,101; U.S. Pat. No. 5,585,089; U.S. Pat. No. 5,693,762 and U.S. Pat. No. 6,180,370). Accordingly, another embodiment of the invention pertains to an isolated antibody, or antigen binding portion thereof, comprising the VH and VL CDR sequences of anti-Ficolin-3 antibody FCN308, FCN334, and FCN329 described herein, yet these antibodies may contain framework sequences different from these antibodies.

The invention also provides a chimeric or humanized version of a murine anti-human Ficolin-3 monoclonal antibody, or antigen-binding fragment thereof, which binds human Ficolin-3, and the use of such antibodies (e.g., in the modulation of human Ficolin-3-mediated physiological processes in a mammalian host). In one embodiment, the murine antibody is one of anti-Ficolin-3 antibody FCN308, FCN334, or FCN329 with framework sequences different from these antibodies. In one embodiment, the humanized antibody is a humanized version of anti-Ficolin-3 antibody FCN308, FCN334, and FCN329 described herein.

Framework sequences can be obtained from public DNA databases or published references that include germline antibody gene sequences. For example, germline DNA sequences for human heavy and light chain variable region genes can be found in the “dBase” human germline sequence database (available on the Internet at www.mrc-cpe.cam.ac.uk/vbase), as well as in Kabat, E. A., et al. (1991) Sequences of Proteins of Immunological Interest, Fifth Edition, U.S. Department of Health and Human Services, NIH Publication No. 91-3242; Tomlinson, I. M., et al. (1992) “The Repertoire of Human Germline VH Sequences Reveals about Fifty Groups of VH Segments with Different Hypervariable Loops” J. Mol. Biol. 227:776-798; and Cox, J. P. L. et al. (1994) “A Directory of Human Germ-line VH Segments Reveals a Strong Bias in their Usage” Eur. J. Immunol. 24:827-836; the contents of each of which are expressly incorporated herein by reference. The VH CDR 1, 2 and 3 sequences of the antibodies according to the invention, and the VL CDR 1, 2 and 3 sequences of the antibodies according to the invention can be grafted onto framework regions that have the same sequence as that found in the germline immunoglobulin gene from which the framework sequence derive, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. No. 5,530,101; U.S. Pat. No. 5,585,089; U.S. Pat. No. 5,693,762 and U.S. Pat. No. 6,180,370).

In another aspect of the invention, the structural features of anti-human Ficolin-3 antibodies of the invention are used to create structurally related anti-human Ficolin-3 antibodies that retain at least one functional property of the antibodies of the invention, such as binding to human Ficolin-3. For example, one or more CDR regions of anti-Ficolin-3 antibody FCN308, FCN334, and FCN329 described herein, or variants thereof, can be combined recombinantly with known framework regions and/or other CDRs to create additional, recombinantly-engineered, anti-human Ficolin-3 antibodies of the invention. The starting material for the engineering method is one or more of the VH and/or VL sequences provided herein, or one or more CDR regions thereof. To create the engineered antibody, it is not necessary to actually prepare (i.e., express as a protein) an antibody having one or more of the VH and/or VL sequences provided herein, or one or more CDR regions thereof. Rather, the information contained in the sequence(s) is used as the starting material to create a “second generation” sequence(s) derived from the original sequence(s) and then the “second generation” sequence(s) is prepared and expressed as a protein.

Another type of variable region modification is to mutate amino acid residues within the VH and/or VL CDR1, CDR2 and/or CDR3 regions to thereby improve one or more binding properties (e.g., affinity) of the antibody of interest. Site-directed mutagenesis or PCR-mediated mutagenesis can be performed to introduce the mutation(s) and the effect on antibody binding, or other functional property of interest, can be evaluated in in vitro or in vivo assays as described herein and provided in the Examples. Preferably conservative modifications (as discussed above) are introduced. The mutations may be amino acid substitutions, additions or deletions. Moreover, typically no more than 8, more typically no more than 5 residues are altered within a single CDR region.

Engineered antibodies of the invention include those in which modifications have been made to framework residues within VH and/or VL, e.g. to improve the properties of the antibody. Typically such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived.

Another type of framework modification involves mutating one or more residues within the framework region, or even within one or more CDR regions, to remove T cell epitopes to thereby reduce the potential immunogenicity of the antibody. This approach is also referred to as “deimmunization” and is described in further detail in US 2003/0153043. Fc modifications In addition or as an alternative to modifications made within the framework or CDR regions, antibodies of the invention may be engineered to include modifications within the Fc region, typically to alter one or more functional properties of the antibody, such as serum half-life, complement fixation, Fc receptor binding, protein stability and/or antigen-dependent cellular cytotoxicity, or lack thereof. Furthermore, an antibody of the invention may be chemically modified (e.g. one or more chemical moieties can be attached to the antibody) or be modified to alter its glycosylation, again to alter one or more functional properties of the antibody. Each of these embodiments is described in further detail below. The residues in the Fc region are numbered according to Kabat.

If desired, the class of an antibody may be “switched” by known techniques. Such techniques include, e.g., the use of direct recombinant techniques (see e.g., U.S. Pat. No. 4,816,397) and cell-cell fusion techniques (see e.g., U.S. Pat. No. 5,916,771). For example, an antibody that was originally produced as an IgM molecule may be class switched to an IgG antibody. Class switching techniques also may be used to convert one IgG subclass to another, e.g., from IgGI to IgG2. Thus, the effector function of the antibodies of the invention may be changed by isotype switching to, e.g., an IgGI, IgG2, IgG3, IgG4, IgD, IgA, IgE, or IgM antibody for various therapeutic uses. Exemplary cDNA sequences for constant regions are available via, e.g., GenBank (accessible via NCBI and other public websites), each of which incorporated by reference in its entirety, are as follows: Human IgGI constant heavy chain region: GenBank accession No.: J00228; Human IgG2 constant heavy chain region: GenBank accession No.: J00230; Human IgG3 constant heavy chain region: GenBank accession No.: X04646; Human IgG4 constant heavy chain region: GenBank accession No.: K01316; and Human kappa light chain constant region: GenBank accession No.: J00241. In one embodiment, the hinge region of CH1 is modified such that the number of cysteine residues in the hinge region is altered, e.g., increased or decreased. This approach is described further in U.S. Pat. No. 5,677,425. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half-life of the antibody. More specifically, one or more amino acid mutations are Introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745. In another embodiment, the antibody is modified to increase its biological half-life. Various approaches are possible. For example, one or more of the following mutations can be introduced: T252L, T254S, and T256F, as described in U.S. Pat. No. 6,277,375. Alternatively, to increase the biological half-life, the antibody can be altered within the CH 1 or CL region to contain a salvage receptor binding epitope taken from two loops of a CH2 domain of an Fc region of an IgG, as described in U.S. Pat. No. 5,869,046 and U.S. Pat. No. 6,121,022. In yet other embodiments, the Fc region is altered by replacing at least one amino acid residue with a different amino acid residue to alter the effecter function of the antibody. For example, one or more amino acids selected from amino acid residues 234, 235, 236, 237, 297, 318, 320 and 322 can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. No. 5,624,821 and U.S. Pat. No. 5,648,260, both to Winter et al. In another example, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Pat. No. 6,194,551|. In another example, one or more amino acid residues within amino acid positions 231 and 239 are altered to thereby alter the ability of the antibody to fix complement. This approach is described further in WO 94/29351. In yet another example, the Fc region is modified to increase the ability of the antibody to mediate antibody dependent cellular cytotoxicity (ADCC) and/or to increase the affinity of the antibody for an Fey receptor by modifying one or more amino acids at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439. This approach is described further in WO 00/42072. Moreover, the binding sites on human IgGI for FcyRI, FcyRII, FcyRI 11 and FcRn have been mapped and variants with improved binding have been described (see Shields, R. L. et al. (2001) J. Biol. Chem. 276:6591-6604). Specific mutations at positions 256, 290, 298, 333, 334 and 339 were shown to improve binding to FcRIII. Additionally, the following combination mutants were shown to improve FcyRI 11 binding: T256A/S298A, S298A/E333A, S298A/K224A and S298A/E333A/K334A.

The constant region may further be modified to stabilize the antibody, e.g., to reduce the risk of a bivalent antibody separating into two monovalent VH-VL fragments. For example, in an IgG4 constant region, residue S241 may be mutated to a proline (P) residue to allow complete disulphide bridge formation at the hinge (see, e.g., Angal et al., Mol Immunol. 1993; 30:105-8).

### Glycosylation Modifications

In still another embodiment, the glycosylation of an antibody is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. No. 5,714,350 and U.S. Pat. No. 6,350,861. Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation “machinery”. Cells with such alterations have been described in the art and can be used as host cells in which to express recombinant antibodies of the invention to thereby produce an antibody with altered glycosylation. For example, EP 1176195 by Hanai et al. describes a cell line with a functionally disrupted FUT8 gene, which encodes a fucosyl transferase, such that antibodies expressed in such a cell line exhibit hypofucosylation. WO 03/035835 describes a variant CHO cell line, Lecl3 cells, with reduced ability to attach fucose to Asn(297)-linked carbohydrates, also resulting in hypofucosylation of antibodies expressed in that host cell (see also Shields, R. L. et al. (2002) J. Biol. Chem. 277:26733-26740). WO 99/54342 describes cell lines engineered to express glyco-protein-modifying glycosyl transferases (e.g., beta(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 7:176-180). In certain embodiments of the methods of engineering antibodies of the invention, mutations can be introduced randomly or selectively along all or part of an anti-human Ficolin-3 antibody coding sequence and the resulting modified antibodies can be screened for binding activity and/or other functional properties as described herein. Mutational methods have been described in the art. For example, WO 02/092780 describes methods for creating and screening antibody mutations using saturation mutagenesis, synthetic ligation assembly, or a combination thereof.

Alternatively, WO 03/074679 describes methods of using computational screening methods to optimize physiochemical properties of antibodies.

### Antibody Derivatives

Antibody derivatives (or immunoconjugates) within the scope of this invention include anti-human Ficolin-3 antibodies conjugated or covalently bound to a second agent.

For example, in one aspect, the invention provides immunoconjugates comprising an antibody conjugated or covalently bonded to a radioactive isotope, such as a therapeutic radionuclide or a radionuclide suitable for detection purposes. Any of a number of suitable radioactive isotopes can be used, including, but not limited to, 1-131, Indium-111, Lutetium-171, Bismuth-212, Bismuth-213, Astatine-211, Copper-62, Copper-64, Copper-67, Yttrium-90, Iodine-125, Iodine-131, Phosphorus-32, Phosphorus-33, Scandium-47, Silver-111, Gallium-67, Praseodymium-142, Samarium-153, Terbium-161, Dysprosium-166, Holmium-166, Rhenium-186, Rhenium-188, Rhenium-189, Lead-212, Radium-223, Actinium-225, Iron-59, Selenium-75, Arsenic-77, Strontium-89, Molybdenum-99, Rhodium-105, Palladium-109, Praseodymium-143, Promethium-149, Erbium-169, Iridium-194, Gold-198, Gold-199, and Lead-211. In general, the radionuclide preferably has a decay energy in the range of 20 to 6,000 keV, preferably in the ranges 60 to 200 keV for an Auger emitter, 100-2,500 keV for a beta emitter, and 4,000-6,000 keV for an alpha emitter. Also preferred are radionuclides that substantially decay with generation of alpha-particles.

The antibody conjugates of the invention can be used to modify a given biological response, where the drug moiety is not to be construed as limited to classical chemical therapeutic agents. For example, the drug moiety may be a protein or polypeptide possessing a desired biological activity.

The second agent can be linked to the antibody directly or indirectly, using any of a large number of available methods. For example, an agent can be attached at the hinge region of the reduced antibody component via disulfide bond formation, using cross-linkers such as N-succinyl 3-(2-pyridyldithio)proprionate (SPDP), or via a carbohydrate moiety in the Fc region of the antibody (see, e.g., Yu et al. (1994) Int. J. Cancer 56: 244; Wong, Chemistry of Protein Conjugation and Cross-linking (CRC Press 1991); Upeslacis et al., “Modification of Antibodies by Chemical Methods,” in Monoclonal antibodies: principles and applications, Birch et al. (eds.), pages 187-230 (Wiley-Liss, Inc. 1995); Price, “Production and Characterization of Synthetic Peptide-Derived Antibodies,” in Monoclonal antibodies: Production, engineering and clinical application, Ritter et al. (eds.), pages 60-84 (Cambridge University Press 1995), Cattel ef a/. (1989) Chemistry today 7:51-58, Delprino et al. (1993) J. Pharm. Sci 82:699-704; Arpicco et al. (1997) Bioconjugate Chemistry 8:3; Reisfeld et al. (1989) Antibody, Immunicon. Radiopharm. 2:217; the entire disclosures of each of which are herein incorporated by reference). See, also, e.g. Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al., (eds.), pp. 303-16 (Academic Press 1985), and Thorpe et al., “The Preparation And Cytotoxic Properties Of Antibody-Toxin Conjugates”, Immunol. Rev., 62:119-58 (1982).

In other embodiments, the second agent is a detectable moiety, which can be any molecule that can be quantitatively or qualitatively observed or measured. Examples of detectable markers useful in the conjugated antibodies of this invention are radioisotopes, fluorescent dyes, or a member of a complementary binding pair, such as a member of any one of: and antigen/antibody (other than an antibody to Ficolin-3), lectin/carbohydrate; avidin/biotin; receptor/ligand; or molecularly imprinted polymer/print molecule systems.

The second agent may also or alternatively be a polymer, intended to, e.g., increase the circulating half-life of the antibody. Exemplary polymers and methods to attach such polymers to peptides are illustrated in, e.g., U.S. Pat. No. 4,766,106; U.S. Pat. No. 4,179,337; U.S. Pat. No. 4,495,285; and U.S. Pat. No. 4,609,546. Additional illustrative polymers include polyoxyethylated polyols and polyethylene glycol (PEG) moieties. As used herein, the term “polyethylene glycol” is intended to encompass any of the forms of PEG that have been used to derivatize other proteins, such as mono(C1-C10) alkoxy- or aryloxy-polyethylene glycol or polyethylene glycol-maleimide. For example, a full-length antibody or antibody fragment can be conjugated to one or more PEG molecules with a molecular weight of between about 1,000 and about 40,000, such as between about 2000 and about 20,000, e.g., about 3,000-12,000. To pegylate an antibody or fragment thereof, the antibody or fragment typically is reacted with polyethylene glycol (PEG), such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). In certain embodiments, the antibody to be pegylated is an aglycosylated antibody. Methods for pegylating proteins are known in the art and can be applied to the antibodies of the Invention. See for example, EP 154316, WO 2004/099231, and EP 401384.

### Nucleic Acids

Another aspect of the invention pertains to nucleic acid molecules that encode the antibodies of the invention. The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. See, F. Ausubel, et al., ed. (1987) Current Protocols in Molecular Biology, Greene Publishing and Wiley Interscience, New York. A nucleic acid of the invention can be, for example, DNA or RNA and may or may not contain intronic sequences. In a preferred embodiment, the nucleic acid is a cDNA molecule. While the following paragraphs refer to DNA sequences or use thereof, the same methods or principles can generally be applied to mRNA sequences.

Nucleic acids of the invention can be obtained using standard molecular biology techniques. For antibodies expressed by hybridomas (e.g., hybridomas prepared from transgenie mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), nucleic acids encoding the antibody can be recovered from the library.

Once DNA fragments encoding VH and VL segments are obtained, these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (CH1, CH2 and CH3). The sequences of human heavy chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG4 constant region.

For a Fab fragment heavy chain gene, the VH-encoding DNA can be operatively linked to another DNA molecule encoding only the heavy chain CH 1 constant region.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region, but most preferably is a kappa constant region. To create a scFv gene, the VH- and VL-encoding DNA fragments are operatively linked to another fragment encoding a flexible linker, e.g., encoding the amino acid sequence (Gly4-Ser)₃, such that the VH and VL sequences can be expressed as a contiguous singlechain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al., (1990) Nature 348:552-554).

### Antibody Production

Monoclonal antibodies (mAbs) of the present invention can be produced by a variety of techniques, including conventional monoclonal antibody methodology e.g., the standard somatic cell hybridization technique of Kohler and Milstein (1975) Nature 256: 495. Although somatic cell hybridization procedures are preferred, in principle, other techniques for producing monoclonal antibody can be employed e.g., viral or oncogenic transformation of B lymphocytes.

One preferred animal system for preparing hybridomas is the murine system. Immunization protocols and techniques for isolation of immunized splenocytes for fusion are known in the art, as are fusion partners (e.g., murine myeloma cells) and fusion procedures. Chimeric or humanized antibodies of the present invention can also be prepared based on the sequence of a murine monoclonal antibody using established techniques. For example, DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567). To create a humanized antibody, the murine CDR regions can be inserted into a human framework using methods known in the art (see e.g., U.S. Pat. No. 5,225,539, U.S. Pat. No. 5,530,101; U.S. Pat. No. 5,585,089; U.S. Pat. No. 5,693,762 and U.S. Pat. No. 6,180,370).

In a preferred embodiment, the antibodies of the invention are human monoclonal antibodies. Such human monoclonal antibodies directed against human Fcolin-3 can be generated using transgenic or transchromosomic mice carrying parts of the human immune system rather than the mouse system. These transgenic and transchromosomic mice include mice referred to herein as HuMAb mice and KM mice, respectively, and are collectively referred to herein as “human Ig mice.” The HuMAb mouse (Medarex, Inc.) contains human immunoglobulin gene miniloci that encode unrearranged human heavy (p and y) and K light chain immunoglobulin sequences, together with targeted mutations that inactivate the endogenous, u and K chain loci (see e.g., Lonberg, et al. (1994) Nature 368: 856-859). Accordingly, the mice exhibit reduced expression of mouse IgM or K, and, in response to immunization, the introduced human heavy and light chain transgenes undergo class switching and somatic mutation to generate high affinity human IgGK monoclonal (Lonberg, N. et al. (1994), supra; reviewed in Lonberg, N. (1994) Handbook of Experimental Pharmacology 113:49-101; Lonberg, N. and Huszar, D. (1995) Intern. Rev. Immunol. 13: 65-93, and Harding, F. and Lonberg, N. (1995) Ann. N.Y. Acad. Sci. 764:536-546). The preparation and use of HuMab mice, and the genomic modifications carried by such mice, is further described in Taylor, L. et al. (1992) Nucleic Acids Research 20:6287-6295; Chen, J. et al. (1993) International Immunology 5: 647-656; Tuaillon et al. (1993) Proc. Natl. Acad. Sci. USA 90:3720-3724; Choi et al. (1993) Nature Genetics 4: 117-123; Chen, J. et al. (1993) EMBO J. 12: 821-830; Tuaillon et al. (1994) J. Immunol. 152:2912-2920; Taylor, L. et al. (1994) International immunology 6: 579-591; and Fishwild, D. et al. (1996) Nature Biotechnology 14: 845-851, the contents of all of which are hereby specifically incorporated by reference in their entirety. See further, U.S. Pat. No. 5,545,806; U.S. Pat. No. 5,569,825; U.S. Pat. No. 5,625,126; U.S. Pat. No. 5,633,425; U.S. Pat. No. 5,789,650; U.S. Pat. No. 5,877,397; U.S. Pat. No. 5,661,016; U.S. Pat. No. 5,814,318; U.S. Pat. No. 5,874,299; and U.S. Pat. No. 5,770,429; U.S. Pat. No. 5,545,807; WO 92/03918, WO 93/12227, WO 94/25585, WO 97/13852, WO 98/24884 and WO 99/45962; and WO 01/14424. In another embodiment, human antibodies of the invention can be raised using a mouse that carries human immunoglobulin sequences on transgenes and transchomosomes, such as a mouse that carries a human heavy chain transgene and a human light chain transchromosome. Such mice, referred to herein as “KM mice”, are described in detail in WO 02/43478. Still further, alternative transgenic animal systems expressing human immunoglobulin genes are available in the art and can be used to raise anti-human Ficolin-3 antibodies of the invention. For example, an alternative transgenic system referred to as the Xenomouse (Abgenix, Inc.) can be used; such mice are described in, for example, U.S. Pat. No. 5,939,598; U.S. Pat. No. 6,075,181; U.S. Pat. No. 6,114,598; U.S. Pat. No. 6,150,584 and U.S. Pat. No. 6,162,963. Moreover, alternative transchromosomic animal systems expressing human immunoglobulin genes are available in the art and can be used to raise anti-human Ficolin-3 antibodies of the invention. For example, mice carrying both a human heavy chain transchromosome and a human light chain tranchromosome, referred to as “TC mice” can be used; such mice are described in Tomizuka et al. (2000) Proc. Natl. Acad. Sci. USA 97:722-727. Furthermore, cows carrying human heavy and light chain transchromosomes have been described in the art (Kuroiwa et al. (2002) Nature Biotechnology 20:889-894) and can be used to raise anti-human Ficolin-3 antibodies of the invention.

Human monoclonal antibodies of the invention can also be prepared using phage display methods for screening libraries of human immunoglobulin genes. Such phage display methods for isolating human antibodies are established in the art. See for example: U.S. Pat. No. 5,223,409; U.S. Pat. No. 5,403,484; U.S. Pat. No. 5,571,698; U.S. Pat. No. 5,427,908 and U.S. Pat. No. 5,580,717; U.S. Pat. No. 5,969,108; U.S. Pat. No. 6,172,197; U.S. Pat. No. 5,885,793; U.S. Pat. No. 6,521,404; U.S. Pat. No. 6,544,731; U.S. Pat. No. 6,555,313; U.S. Pat. No. 6,582,915 and U.S. Pat. No. 6,593,081. Human monoclonal antibodies of the invention can also be prepared using SCID mice into which human immune cells have been reconstituted such that a human antibody response can be generated upon immunization. Such mice are described in, for example, U.S. Pat. No. 5,476,996 and U.S. Pat. No. 5,698,767.

When human Ig mice are used to raise human antibodies of the invention, such mice can be immunized with a purified or enriched preparation of human Ficolin-3 antigen and/or cells expressing human Ficolin-3, as described by Lonberg, N. et al. (1994) Nature 368(6474): 856-859; Fishwild, D. et al. (1996) NatureBiotechnology 14: 845-851; and WO 98/24884 and WO 01/14424. Preferably, the mice will be 6-16 weeks of age upon the first infusion. For example, a purified or enriched preparation (5-50 μg) of human Ficolin-3 antigen can be used to immunize the human Ig mice intraperitoneal. In the event that immunizations using a purified or enriched preparation of human Ficolin-3 antigen do not result in antibodies, mice can also be immunized with cells expressing human Ficolin-3, e.g., a human NK or T-cell line, or a mammalian cell expressing recombinant human Ficolin-3 with or without DAP10, to promote immune responses.

Detailed procedures to generate fully human monoclonal antibodies to human Ficolin-3 are described herein. The form and amount of antigen administered (e.g., human Ficolin-3 polypeptide or cell expressing human Ficolin-3), as well as administration schedules and the possible use of adjuvants such as, e.g., complete Freund's adjuvant or incomplete Freund's adjuvant, are typically optimized for each antigen-mouse system according to established methods in the art.

The immune response can be monitored over the course of the immunization protocol with plasma samples being obtained by retroorbital bleeds, and the plasma or serum can be screened by ELISA (as described below), and mice with sufficient titers of anti-human Ficolin-3 human immunoglobulin can be used for fusions. Mice can be boosted intravenously with antigen 3 days before sacrifice and removal of the spleen. It is expected that 2-3 fusions for each immunization may need to be performed.

To generate hybridomas producing human monoclonal antibodies of the invention, splenocytes and/or lymph node cells from immunized mice can be isolated and fused to an appropriate immortalized cell line, such as a mouse myeloma cell line. The resulting hybridomas can be screened for the production of antigen-specific antibodies. For example, single cell suspensions of splenic lymphocytes from immunized mice can be fused to one-sixth the number of P3×63-Ag8.653 nonsecreting mouse myeloma cells (ATCC, CRL 1580) with 50% PEG. Alternatively, the cells can be fused by electrofusion. Cells are plated at approximately 2×105 in a flat bottom microtiter plate, followed by a two week incubation in selective medium containing 20% fetal Clone Serum, 18% “653” conditioned media, 5% origen (IGEN), 4 mM L-glutamine, 1 mM sodium pyruvate, 5 mM HEPES, 0.055 mM 2-mercaptoethanol, 50 units/ml penicillin, 50 mg/ml streptomycin, 50 mg/ml gentamycin and 1×HAT (Sigma; the HAT is added 24 hours after the fusion). After approximately two weeks, cells can be cultured in medium in which the HAT is replaced with HT. Individual wells can then be screened by ELISA for human monoclonal IgM and IgG antibodies. Once extensive hybridoma growth occurs, medium can be observed usually after 10-14 days. The antibody secreting hybridomas can be replated, screened again, and if still positive for human IgG, the monoclonal antibodies can be subcloned at least twice by limiting dilution. The stable subclones can then be cultured in vitro to generate small amounts of antibody in tissue culture medium for characterization. To purify human monoclonal antibodies, selected hybridomas can be grown in two-liter spinner-flasks for monoclonal antibody purification. Supernatants can be filtered and concentrated before affinity chromatography with protein A-sepharose (Pharmacia, Piscataway, N.J.). Eluted IgG can be checked by gel electrophoresis and high performance liquid chromatography to ensure purity. The buffer solution can be exchanged into PBS, and the concentration can be determined by spectroscopy. The monoclonal antibodies can be aliquoted and stored at −80°

Antibodies of the invention can also be produced in a host cell transfectoma using, for example, a combination of recombinant DNA techniques and gene transfection methods as is well known in the art (e.g., Morrison, S. (1985) Science 229:1202).

For example, to express the antibodies, DNAs encoding partial or full-length light and heavy chains, can be obtained by standard molecular biology techniques (e.g. PCR amplification or cDNA cloning using a hybridoma that expresses the antibody of interest) and the DNAs can be inserted into expression vectors such that the genes are operatively linked to transcriptional and translational control sequences and may serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). The light and heavy chain variable regions of the antibodies described herein can be used to create full length antibody genes of any antibody isotype by inserting them into expression vectors already encoding heavy chain constant and light chain constant regions of the desired isotype such that the VH segment is operatively linked to the CH segment(s) within the vector and the VL segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. The term “regulatory sequence” is intended to include promoters, enhancers and other expression control elements (e.g. polyadenylation signals) that control the transcription or translation of the antibody chain genes. Such regulatory sequences are described, for example, in Goeddel (Gene Expression Technology. Methods in Enzymology 185, Academic Press, San Diego, Calif. (1990)).

It will be appreciated by those skilled in the art that the design of the expression vector, including the selection of regulatory sequences, may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP) and polyoma. Alternatively, nonviral regulatory sequences may be used, such as the ubiquitin promoter or p-globin promoter. Still further, regulatory elements composed of sequences from different sources, such as the SRa promoter system, which contains sequences from the SV40 early promoter and the long terminal repeat of human T cell leukemia virus type 1 (Takebe, Y. et al. (1988) Mol. Cell. Biol. 8:466-472). In addition to the antibody chain genes and regulatory sequences, the recombinant expression vectors of the invention may carry additional sequences, such as sequences that regulate replication of the vector in host cells (e.g. origins of replication) and selectable marker genes. The selectable marker gene facilitates selection of host cells into which the vector has been introduced (see, e.g. U.S. Pat. No. 4,399,216, U.S. Pat. No. 4,634,665 and U.S. Pat. No. 5,179,017). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection). For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss, M. A. and Wood, C. R. (1985) Immunology Today 6:12-13).

Preferred mammalian host cells for expressing the recombinant antibodies of the invention include Chinese Hamster Ovary (CHO cells) (including dhfr-CHO cells, described in Urlaub and Chasin, (1980) Proc. Nail. Acad. Sci. USA 77:4216-4220, used with a DHFR selectable marker, e.g., as described in R. J. Kaufman and P. A. Sharp (1982) Mol. Biol. 159:601-621), NSO myeloma cells, COS cells and SP2 cells. In particular, for use with NSO myeloma cells, another preferred expression system is the GS gene expression system disclosed in WO 87/04462, WO 89/01036 and EP 338841. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

### Antibody Characterization

After production or purification, or as part of a screening or selection procedure, the functional characteristics of an anti-human Ficolin-3 antibody of the invention can be investigated. Functional properties of interest include, e.g., antibody binding specificity for human Ficolin-3, antibody competition with human Ficolin-3-ligands, antibody competition with reference antibodies (such as anti-Ficolin-3 antibody FCN308, FCN334, and FCN329 described herein), the epitope to which the antibody binds, the affinity of the antibody-antigen interaction, and antagonistic/agonistic properties of the antibody.

The following are brief descriptions of exemplary assays for antibody characterization. Some are further described in subsequent sections and/or described in the Examples.

(1) Antibody specificity for human Ficolin-3 can be evaluated by confirming that the monoclonal antibody (or, as part of animal screening procedures, serum containing polyclonal antibodies) block binding of human ficolin-3 to its ligands. Ligand binding may be evaluated by any suitable competition assay.
(2) Affinity parameters, including on- and off-rate, of antibodies can determined on a Biacore machine. For example, human Ficolin-3-Fc protein can be immobilized on a chip, the antibody passed over the chip, the on- and off-rates determined, and the KD calculated.
(3) The ability of an antibody to block human Ficolin-3-ligand mediated complement activation.

The present invention provides for antibodies, and antigen-binding fragments and immunoconjugates thereof, that bind human Ficolin-3. Any of a wide variety of assays can be used to assess binding of an antibody to human Ficolin-3. Protocols based upon ELISAs, radioimmunoassays, Western blotting, BIACORE, and other competition assays, inter alia, are suitable for use and are well known in the art. Further, several binding assays, including competition assays, are described in the Examples.

For example, simple binding assays can be used, in which a test antibody is incubated in the presence of a target protein or epitope (e.g., Ficolin-3 or a portion thereof), unbound antibodies are washed off, and the presence of bound antibodies is assessed using, e.g., radiolabels, physical methods such as mass spectrometry, or direct or indirect fluorescent labels detected using, e.g., cytofluorometric analysis (e.g. FACScan). Such methods are well known to those of skill in the art. Any amount of binding above the amount seen with a control, non-specific antibody indicates that the antibody binds specifically to the target.

In such assays, the ability of the test antibody to bind to human Ficolin-3 can be compared with the ability of a (negative) control protein, e.g. an antibody raised against a structurally unrelated antigen, or a non-Ig peptide or protein, to bind to the same target. Antibodies or fragments that bind to Ficolin-3 using any suitable assay with 25%, 50%, 100%, 200%, 1000%, or higher increased affinity relative to the control protein, are said to “specifically bind to” or “specifically interact with” the target, and are preferred for use in the therapeutic methods described below. The ability of a test antibody to affect the binding of a (positive) control antibody against Ficolin-3, e.g. anti-Ficolin-3 antibody FCN308, FCN334, and FCN329 described herein, may also be assessed.

In one aspect, the invention provides for anti-human Ficolin-3 antibodies sharing biological characteristics and/or substantial VH and/or VL sequence identity with anti-Ficolin-3 antibody FCN308, FCN334, and FCN329 described herein. One exemplary biological characteristic is the binding to the epitope of anti-Ficolin-3 antibody FCN308, FCN334, and FCN329 described herein, i.e., the respective regions in the extracellular domain of human Ficolin-3 to which the FCN308, FCN334, and/or FCN329 antibodies bind. To screen for antibodies that bind to the FCN308, FCN334, and/or FCN329 epitope, a routine cross-blocking assay, such as that described in Antibodies, A Laboratory Manual, Cold Spring Harbor Laboratory, Ed Harlow and David Lane (1988), can be performed. In an exemplary cross-blocking or competition assay, FCN308, FCN334, and/or FCN329 antibody and a test antibody are admixed (or pre-adsorbed) and applied to a sample containing Ficolin-3. In certain embodiments, one would pre-mix the control antibodies with varying amounts of the test antibody (e.g., 1:10 or 1:100) for a period of time prior to applying to the Ficolin-3-containing sample. In other embodiments, the control and varying amounts of test antibody can simply be admixed during exposure to the antigen/target sample. As long as one can distinguish bound from free antibodies (e.g., by using separation or washing techniques to eliminate unbound antibodies) and the control antibody from test antibody (e.g., by using species- or isotype-specific secondary antibodies, by specifically labeling the control antibody with a detectable label, or by using physical methods such as mass spectrometry to distinguish between different compounds) one will be able to determine if the test antibody reduces the binding of the control antibody to the antigen, indicating that the test antibody recognizes substantially the same epitope as the control. In this assay, the binding of the (labeled) control antibody in the presence of a completely irrelevant antibody is the control high value. The control low value is be obtained by incubating the labeled (positive) control antibody with unlabeled control antibody, where competition would occur and reduce binding of the labeled antibody.

In a test assay, a significant reduction in labeled antibody reactivity in the presence of a test antibody is indicative of a test antibody that recognizes the same epitope, i.e., one that “cross-reacts” with the labeled control antibody. Any test antibody or compound that reduces the binding of the labeled control to the antigen/target by at least 50% or more preferably 70%, at any ratio of control:test antibody or compound between about 1:10 and about 1:100 is considered to be an antibody or compound that binds to substantially the same epitope or determinant as the control. Preferably, such test antibody or compound will reduce the binding of the control to the antigen/target by at least 90%. Nevertheless, any compound or antibody that reduces the binding of a control antibody or compound to any measurable extent can be used in the present invention.

### Functional Assays

Description of in vitro complement inhibition assays are described in the examples.

### Pharmaceutical Formulations

In one embodiment, the present invention provides a pharmaceutical composition or formulation comprising anti-human Ficolin-3 antibodies as described herein together with one or more carriers.

Accordingly, one exemplary aspect of the invention is a pharmaceutical formulation comprising such an antibody which is present in a concentration from 1 mg/ml to 500 mg/ml, and wherein said formulation has a pH from 2.0 to 10.0. The formulation may further comprise a buffer system, preservative(s), tonicity agent(s), chelating agent(s), stabilizers, and/or surfactants. In one embodiment, the pharmaceutical formulation is an aqueous formulation, i.e., formulation comprising water. Such formulation is typically a solution or a suspension. In a further embodiment, the pharmaceutical formulation is an aqueous solution. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In another embodiment, the pharmaceutical formulation is a freeze-dried formulation, whereto the physician or the patient may add solvents and/or diluents prior to administration.

In another embodiment, the pharmaceutical formulation is a dried formulation (e.g. freeze-dried or spray-dried) ready for use without any prior dissolution.

In a further aspect, the pharmaceutical formulation comprises an aqueous solution of such an antibody, and a buffer, wherein the antibody is present in a concentration from 1 mg/ml or above, and wherein said formulation has a pH from about 2.0 to about 10.0.

In another embodiment, the pH of the formulation is in the range selected from the list consisting of from about 2.0 to about 10.0, about 3.0 to about 9.0, about 4.0 to about 8.5, about 5.0 to about 8.0, and about 5.5 to about 7.5.

In a further embodiment, the formulation includes a buffer that is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative embodiment of the invention.

In a further embodiment, the formulation also or alternatively comprises a pharmaceutically acceptable preservative. The preservative may be selected from, e.g., the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) or mixtures thereof. The preservative may, e.g., be present in a concentration from 0.1 mg/ml to 20 mg/ml, from 0.1 mg/ml to 5 mg/ml, from 5 mg/ml to 10 mg/ml, or from 10 mg/ml to 20 mg/ml. Each one of these specific preservatives constitutes an alternative embodiment of the invention. The use of a preservative in pharmaceutical compositions is well-known to the skilled person. For convenience reference is made to Remington: The Science and Practice of Pharmacy, 19th edition, 1995. In a further embodiment, the formulation also or alternatively comprises an isotonic agent. The isotonic agent may be, e.g., selected from the group consisting of a salt (e.g. sodium chloride), a sugar or sugar alcohol, an amino acid (e.g. L-glycine, L-histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol) polyethyleneglycol (e.g. PEG400), or mixtures thereof. Any sugar such as mono-, di-, or polysaccharides, or water-soluble glucans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used. In one embodiment, the sugar additive is sucrose. Sugar alcohol is defined as a C4-C8 hydrocarbon having at least one —OH group and includes, for example, mannitol, sorbitol, inositol, galactitol, dulcitol, xylitol, and arabitol. In one embodiment, the sugar alcohol additive is mannitol. The sugars or sugar alcohols mentioned above may be used individually or in combination. There is no fixed limit to the amount used, as long as the sugar or sugar alcohol is soluble in the liquid preparation and does not adversely effect the stabilizing effects achieved using the methods of the invention. The sugar or sugar alcohol concentration can, e.g., be between about 1 mg/ml and about 150 mg/ml. The isotonic agent can be present in a concentration from, e.g., 1 mg/ml to 50 mg/ml, from 1 mg/ml to 7 mg/ml, from 8 mg/ml to 24 mg/ml, or from 25 mg/ml to 50 mg/ml. Each one of these specific isotonic agents constitutes an alternative embodiment of the invention. The use of an isotonic agent in pharmaceutical compositions is well-known to the skilled person. For convenience reference is made to Remington: The Science and Practice of Pharmacy, 19th edition, 1995.

In a further embodiment, the formulation also or alternatively comprises a chelating agent. The chelating agent can, for example, be selected from salts of ethylenediamine-tetraacetic acid (EDTA), citric acid, and aspartic acid, and mixtures thereof. The chelating agent may, for example, be present in a concentration from 0.1 mg/ml to 5 mg/ml, from 0.1 mg/ml to 2 mg/ml, or from 2 mg/ml to 5 mg/ml. Each one of these specific chelating agents constitutes an alternative embodiment of the invention. The use of a chelating agent in pharmaceutical compositions is well-known to the skilled person. For convenience reference is made to Remington: The Science and Practice of Pharmacy, 19th edition, 1995. In a further embodiment of the invention the formulation also or alternatively comprises a stabilizer. The use of a stabilizer in pharmaceutical compositions is well-known to the skilled person. For convenience reference is made to Remington: The Science and Practice of Pharmacy, 19th edition, 1995. More particularly, compositions of the invention can be stabilized liquid pharmaceutical compositions whose therapeutically active components include a polypeptide that possibly exhibits aggregate formation during storage in liquid pharmaceutical formulations. By “aggregate formation” is intended a physical interaction between the polypeptide molecules that results in formation of oligomers, which may remain soluble, or large visible aggregates that precipitate from the solution. By “during storage” is intended a liquid pharmaceutical composition or formulation once prepared, is not immediately administered to a subject. Rather, following preparation, it is packaged for storage, either in a liquid form, in a frozen state, or in a dried form for later reconstitution into a liquid form or other form suitable for administration to a subject. By “dried form” is intended the liquid pharmaceutical composition or formulation is dried either by freeze drying (i.e., lyophilization; see, for example, Williams and PoIIi (1984) J. Parenteral Sci. Technol. 38:48-59), spray drying (see Masters (1991) in Spray-Drying Handbook (5th ed; Longman Scientific and Technical, Essez, U.K.), PP-491-676; Broadhead et al. (1992) Drug Devel. Ind. Pharm. 18:1169-1206; and Mumenthaler et al. (1994) Pharm. Res. 11:12-20), or air drying (Carpenter and Crowe (1988) Cryobiology 25:459-470; and Roser (1991) Biopharm. 4:47-53). Aggregate formation by a polypeptide during storage of a liquid pharmaceutical composition can adversely affect biological activity of that polypeptide, resulting in loss of therapeutic efficacy of the pharmaceutical composition. Furthermore, aggregate formation may cause other problems such as blockage of tubing, membranes, or pumps when the polypeptide-containing pharmaceutical composition is administered using an infusion system.

The pharmaceutical compositions of the invention may alternatively or further comprise an amount of an amino acid base sufficient to decrease aggregate formation by the polypeptide during storage of the composition. By “amino acid base” is intended an amino acid or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. In one embodiment, amino acids to use in preparing the compositions of the invention are those carrying a charged side chain, such as arginine, lysine, aspartic acid, and glutamic acid. Any stereoisomer (i.e., L, D, or a mixture thereof) of a particular amino acid (e.g. methionine, histidine, imidazole, arginine, lysine, isoleucine, aspartic acid, tryptophan, threonine and mixtures thereof) or combinations of these stereoisomers, may be present in the pharmaceutical compositions of the invention so long as the particular amino acid is present either in its free base form or its salt form. In one embodiment the L-stereoisomer is used. Compositions of the invention may also be formulated with analogues of these amino acids. By “amino acid analogue” is intended a derivative of the naturally occurring amino acid that brings about the desired effect of decreasing aggregate formation by the polypeptide during storage of the liquid pharmaceutical compositions of the invention. Suitable arginine analogues include, for example, aminoguanidine, ornithine and N-monoethyl L-arginine, suitable methionine analogues include ethionine and buthionine and suitable cysteine analogues include S-methyl-L cysteine. As with the other amino acids, the amino acid analogues are incorporated into the compositions in either their free base form or their salt form. In a further embodiment of the invention the amino acids or amino acid analogues are used in a concentration, which is sufficient to prevent or delay aggregation of the protein.

In a further embodiment of the invention methionine (or other sulphuric amino acids or amino acid analogous) may be added to inhibit oxidation of methionine residues to methionine sulfoxide when the polypeptide acting as the therapeutic agent is a polypeptide comprising at least one methionine residue susceptible to such oxidation. The term “Inhibit” in this context is intended to mean minimal accumulation of methionine oxidized species over time. Inhibiting methionine oxidation results in greater retention of the polypeptide in its proper molecular form. Any stereoisomer of methionine (L or D) or combinations thereof can be used. The amount to be added should be an amount sufficient to inhibit oxidation of the methionine residues such that the amount of methionine sulfoxide is acceptable to regulatory agencies. Typically, this means that the composition contains no more than about 10% to about 30% methionine sulfoxide. Generally, this can be achieved by adding methionine such that the ratio of methionine added to methionine residues ranges from about 1:1 to about 1000:1, such as 10:1 to about 100:1.

In a further embodiment, the formulation further or alternatively comprises a stabilizer selected from the group of high molecular weight polymers or low molecular compounds. In a further embodiment of the invention the stabilizer is selected from polyethylene glycol (e.g. PEG 3350), polyvinyl alcohol (PVA), polyvinylpyrrolidone, carboxy-/hydroxycellulose or derivates thereof (e.g. HPC, HPC-SL, HPC-L and HPMC), cyclodextrins, sulphur-containing substances as monothioglycerol, thioglycolic acid and 2-methylthioethanol, and different salts (e.g. sodium chloride). Each one of these specific stabilizers constitutes an alternative embodiment of the invention.

The pharmaceutical compositions may also or alternatively comprise additional stabilizing agents, which further enhance stability of a therapeutically active polypeptide therein. Stabilizing agents of particular interest to the present invention include, but are not limited to, methionine and EDTA, which protect the polypeptide against methionine oxidation, and a nonionic surfactant, which protects the polypeptide against aggregation associated with freeze-thawing or mechanical shearing. In a further embodiment, the formulation further or alternatively comprises a surfactant. The surfactant may, for example, be selected from a detergent, ethoxylated castor oil, polyglycolyzed glycerides, acetylated monoglycerides, sorbitan fatty acid esters, polyoxypropylene-polyoxyethylene block polymers (eg. poloxamers such as Pluronic® F68, poloxamer 188 and 407, Triton X-100), polyoxyethylene sorbitan fatty acid esters, polyoxyethylene and polyethylene derivatives such as alkylated and alkoxylated derivatives (tweens, e.g. Tween-20, Tween-40, Tween-80 and Brij-35), monoglycerides or ethoxylated derivatives thereof, diglycerides or polyoxyethylene derivatives thereof, alcohols, glycerol, lectins and phospholipids (eg. phosphatidyl serine, phosphatidyl choline, phosphatidyl ethanolamine, phosphatidyl inositol, diphosphatidyl glycerol and sphingomyelin), derivates of phospholipids (eg. dipalmitoyl phosphatidic acid) and lysophospholiplds (eg. palmitoyl lysophosphatidyl-L-serine and 1-acyl-sn-glycero-3-phosphate esters of ethanolamine, choline, serine or threonine) and alkyl, alkoxyl(alkyl ester), alkoxy(alkyl ether)-derivatives of lysophosphatidyl and phosphatidylcholines, e.g. lauroyl and myristoyl derivatives of lysophosphatidylcholine, dipalmitoylphos-phatidylcholine, and modifications of the polar head group, that is cholines, ethanolamines, phosphatidic acid, serines, threonines, glycerol, inositol, and the positively charged DODAC, DOTMA, DCP, BISHOP, lysophosphatidylserine and lysophosphatidylthreonine, and glycerophospholipids (eg. cephalins), glyceroglycolipids (eg. galactopyransoide), sphingoglycolipids (eg. ceramides, gangliosides), dodecylphosphocholine, hen egg lysolecithin, fusidic acid derivatives—(e.g. sodium tauro-dihydrofusidate etc.), long-chain fatty acids and salts thereof C6-C12 (e.g., oleic acid and caprylic acid), acylcarnitines and derivatives, NT acylated derivatives of lysine, arginine or histidine, or side-chain acylated derivatives of lysine or arginine, NT-acylated derivatives of dipeptides comprising any combination of lysine, arginine or histidine and a neutral or acidic amino acid, Nα-acylated derivative of a tripeptide comprising any combination of a neutral amino acid and two charged amino acids, DSS (docusate sodium, CAS registry no [577-11-7]), docusate calcium, CAS registry no [128-49-4]), docusate potassium, CAS registry no [7491-09-0]), SDS (sodium dodecyl sulphate or sodium lauryl sulphate), sodium caprylate, cholic acid or derivatives thereof, bile acids and salts thereof and glycine or taurine conjugates, ursodeoxycholic acid, sodium cholate, sodium deoxycholate, sodium taurocholate, sodium glycocholate, N-Hexadecyl-N,N-dimethyl-3-ammonio-1-propanesulfonate, anionic (alkyl-aryl-sulphonates) monovalent surfactants, zwitterionic surfactants (e.g. N-alkyl-N,N-dimethylammonio-1-propanesulfonates, 3-cholamido-1-propyldimethylammonio-1-propanesulfonate, cationic surfactants (quaternary ammonium bases) (e.g. cetyl-trimethylammonium bromide, cetylpyridinium chloride), non-ionic surfactants (eg. Dodecyl β-D-glucopyranoside), poloxamines (eg. Tetronic's), which are tetrafunctional block copolymers derived from sequential addition of propylene oxide and ethylene oxide to ethylenediamine, or the surfactant may be selected from the group of imidazoline derivatives, or mixtures thereof. Each one of these specific surfactants constitutes an alternative embodiment of the invention.

The use of a surfactant in pharmaceutical compositions is well-known to the skilled person. For convenience reference is made to Remington: The Science and Practice of Pharmacy, 19th edition, 1995.

In a further embodiment, the formulation further or alternatively comprises protease inhibitors such as EDTA (ethylenediamine tetraacetic acid) and benzamidineHCI, but other commercially available protease inhibitors may also be used. The use of a protease inhibitor is particular useful in pharmaceutical compositions comprising zymogens of proteases in order to inhibit autocatalysis.

It is possible that other ingredients may also or alternatively be present in the peptide pharmaceutical formulation of the present invention. Such additional ingredients may include wetting agents, emulsifiers, antioxidants, bulking agents, tonicity modifiers, chelating agents, metal ions, oleaginous vehicles, proteins (e.g., human serum albumin, gelatine or proteins) and a zwitterion (e.g., an amino acid such as betaine, taurine, arginine, glycine, lysine and histidine). Such additional ingredients, of course, should not adversely affect the overall stability of the pharmaceutical formulation of the present invention. Pharmaceutical compositions containing an antibody according to the present invention may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen.

Administration of pharmaceutical compositions according to the invention may be through several routes of administration, for example, lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatment.

Compositions of the current invention may be administered in several dosage forms, for example, as solutions, suspensions, emulsions, microemulsions, multiple emulsion, foams, salves, pastes, plasters, ointments, tablets, coated tablets, rinses, capsules, for example, hard gelatine capsules and soft gelatine capsules, suppositories, rectal capsules, drops, gels, sprays, powder, aerosols, inhalants, eye drops, ophthalmic ointments, ophthalmic rinses, vaginal pessaries, vaginal rings, vaginal ointments, injection solution, in situ transforming solutions, for example in situ gelling, in situ setting, in situ precipitating, in situ crystallization, infusion solution, and implants. Compositions of the invention may further be compounded in, or attached to, for example through covalent, hydrophobic and electrostatic interactions, a drug carrier, drug delivery system and advanced drug delivery system in order to further enhance stability of the antibody, increase bioavailability, increase solubility, decrease adverse effects, achieve chronotherapy well known to those skilled in the art, and increase patient compliance or any combination thereof. Examples of carriers, drug delivery systems and advanced drug delivery systems include, but are not limited to, polymers, for example cellulose and derivatives, polysaccharides, for example dextran and derivatives, starch and derivatives, polyvinyl alcohol), acrylate and methacrylate polymers, polylactic and polyglycolic acid and block copolymers thereof, polyethylene glycols, carrier proteins, for example albumin, gels, for example, thermogelling systems, for example block co-polymeric systems well known to those skilled in the art, micelles, liposomes, microspheres, nanoparticulates, liquid crystals and dispersions thereof, L2 phase and dispersions there of, well known to those skilled in the art of phase behaviour in lipid-water systems, polymeric micelles, multiple emulsions, self-emulsifying, self-microemulsifying, cyclodextrins and derivatives thereof, and dendrimers. Compositions of the current invention are useful in the formulation of solids, semisolids, powder and solutions for pulmonary administration of an antibody, using, for example a metered dose inhaler, dry powder inhaler and a nebulizer, all being devices well known to those skilled in the art.

Compositions of the current invention are specifically useful in the formulation of controlled, sustained, protracting, retarded, and slow release drug delivery systems. More specifically, but not limited to, compositions are useful in formulation of parenteral controlled release and sustained release systems (both systems leading to a many-fold reduction in number of administrations), well known to those skilled in the art. Even more preferably, are controlled release and sustained release systems administered subcutaneous. Without limiting the scope of the invention, examples of useful controlled release system and compositions are hydrogels, oleaginous gels, liquid crystals, polymeric micelles, microspheres, nanoparticles,

Methods to produce controlled release systems useful for compositions of the current Invention include, but are not limited to, crystallization, condensation, co-crystallization, precipitation, co-precipitation, emulsification, dispersion, high pressure homogenisation, encapsulation, spray drying, microencapsulating, coacervation, phase separation, solvent evaporation to produce microspheres, extrusion and supercritical fluid processes. General reference is made to Handbook of Pharmaceutical Controlled Release (Wise, D. L., ed. Marcel Dekker, New York, 2000) and Drug and the Pharmaceutical Sciences vol. 99: Protein Formulation and Delivery (MacNally, E. J., ed. Marcel Dekker, New York, 2000).

Parenteral administration may be performed by subcutaneous, intramuscular, intraperitoneal or intravenous injection by means of a syringe, optionally a pen-like syringe. Alternatively, parenteral administration can be performed by means of an infusion pump. A further option is a composition which may be a solution or suspension for the administration of the antibody compound in the form of a nasal or pulmonal spray. As a still further option, the pharmaceutical compositions containing an antibody of the invention can also be adapted to transdermal administration, e.g. by needle-free injection or from a patch, optionally an iontophoretic patch, or transmucosal, e.g. buccal, administration.

The antibody can be administered via the pulmonary route in a vehicle, as a solution, suspension or dry powder using any of known types of devices suitable for pulmonary drug delivery. Examples of these comprise of, but are not limited to, the three general types of aerosol-generating for pulmonary drug delivery, and may include jet or ultrasonic nebulizers, metered-dose inhalers, or dry powder inhalers (Cf. Yu J, Chien Y W. Pulmonary drug delivery: Physiologic and mechanistic aspects. Crit Rev Ther Drug Carr Sys 14(4) (1997) 395-453).

Based on standardised testing methodology, the aerodynamic diameter (da) of a particle is defined as the geometric equivalent diameter of a reference standard spherical particle of unit density (1 g/cm3). In the simplest case, for spherical particles, da is related to a reference diameter (d) as a function of the square root of the density ratio as described by:

Modifications to this relationship occur for non-spherical particles (cf. Edwards D A, Ben-Jebria A, Langer R. Recent advances in pulmonary drug delivery using large, porous inhaled particles. J Appl Physiol 84(2) (1998) 379-385). The terms “MMAD” and “MMEAD” are well-described and known to the art (cf. Edwards D A, Ben-Jebria A, Langer R and represents a measure of the median value of an aerodynamic particle size distribution. Recent advances in pulmonary drug delivery using large, porous inhaled particles. J Appl Physiol 84(2) (1998) 379-385). Mass median aerodynamic diameter (MMAD) and mass median effective aerodynamic diameter (MMEAD) are used inter-changeably, are statistical parameters, and empirically describe the size of aerosol particles in relation to their potential to deposit in the lungs, independent of actual shape, size, or density (cf. Edwards D A, Ben-Jebria A, Langer R. Recent advances in pulmonary drug delivery using large, porous inhaled particles. J Appl Physiol 84(2) (1998) 379-385). MMAD is normally calculated from the measurement made with impactors, an instrument that measures the particle inertial behaviour in air.

In a further embodiment, the formulation could be aerosolized by any known aerosolisation technology, such as nebulisation, to achieve a MMAD of aerosol particles less than 10 μm, more preferably between 1-5 μm, and most preferably between 1-3 μm. The preferred particle size is based on the most effective size for delivery of drug to the deep lung, where protein is optimally absorbed (cf. Edwards D A, Ben-Jebria A, Langer A, Recent advances in pulmonary drug delivery using large, porous inhaled particles. J Appl Physiol 84(2) (1998) 379-385).

Deep lung deposition of the pulmonal formulations comprising the antibody may optional be further optimized by using modifications of the inhalation techniques, for example, but not limited to: slow inhalation flow (eg. 30 L/min), breath holding and timing of actuation. The term “stabilized formulation” refers to a formulation with increased physical stability, increased chemical stability or increased physical and chemical stability.

The term “physical stability” of the protein formulation as used herein refers to the tendency of the antibody to form biologically inactive and/or insoluble aggregates as a result of exposure of the antibody to thermo-mechanical stresses and/or interaction with interfaces and surfaces that are destabilizing, such as hydrophobic surfaces and interfaces. Physical stability of the aqueous antibody formulations is evaluated by means of visual inspection and/or turbidity measurements after exposing the formulation filled in suitable containers (e.g. cartridges or vials) to mechanical/physical stress (e.g. agitation) at different temperatures for various time periods. Visual inspection of the formulations is performed in a sharp focused light with a dark background. The turbidity of the formulation is characterized by a visual score ranking the degree of turbidity for instance on a scale from 0 to 3 (a formulation showing no turbidity corresponds to a visual score 0, and a formulation showing visual turbidity in daylight corresponds to visual score 3). A formulation is classified physical unstable with respect to antibody aggregation, when it shows visual turbidity in daylight. Alternatively, the turbidity of the formulation can be evaluated by simple turbidity measurements well known to the skilled person. Physical stability of the aqueous antibody formulations can also be evaluated by using a spectroscopic agent or probe of the conformational status of the antibody. The probe is preferably a small molecule that preferentially binds to a non-native conformer of the antibody. One example of a small molecular spectroscopic probe of protein structure is Thioflavin T. Thioflavin T is a fluorescent dye that has been widely used for the detection of amyloid fibrils. In the presence of fibrils, and perhaps other protein configurations as well, Thioflavin T gives rise to a new excitation maximum at about 450 nm and enhanced emission at about 482 nm when bound to a fibril protein form. Unbound Thioflavin T is essentially non-fluorescent at the wavelengths.

Other small molecules can be used as probes of the changes in protein structure from native to non-native states. For instance the “hydrophobic patch” probes that bind preferentially to exposed hydrophobic patches of a protein. The hydrophobic patches are generally buried within the tertiary structure of a protein in its native state, but become exposed as a protein begins to unfold or denature. Examples of these small molecular, spectroscopic probes are aromatic, hydrophobic dyes, such as anthracene, acridine, phenanthroline or the like. Other spectroscopic probes are metal-amino acid complexes, such as cobalt metal complexes of hydrophobic amino acids, such as phenylalanine, leucine, isoleucine, methionine, and valine, or the like.

The term “chemical stability” of the antibody formulation as used herein refers to chemical covalent changes in the antibody structure leading to formation of chemical degradation products with potential less biological potency and/or potential increased immunogenic properties compared to the native antibody structure. Various chemical degradation products can be formed depending on the type and nature of the native antibody and the environment to which the antibody is exposed. Elimination of chemical degradation can most probably not be completely avoided and increasing amounts of chemical degradation products is often seen during storage and use of the antibody formulation as well-known by the person skilled in the art. Most proteins are prone to deamidation, a process in which the side chain amide group in glutaminyl or asparaginyl residues is hydrolysed to form a free carboxylic acid. Other degradations pathways involves formation of high molecular weight transformation products where two or more protein molecules are covalently bound to each other through transamidation and/or disulfide interactions leading to formation of covalently bound dimer, oligomer and polymer degradation products (Stability of Protein Pharmaceuticals, Ahern. T. J. & Manning M. C, Plenum Press, New York 1992). Oxidation (of for instance methionine residues) can be mentioned as another variant of chemical degradation. The chemical stability of the antibody formulation can be evaluated by measuring the amount of the chemical degradation products at various time-points after exposure to different environmental conditions (the formation of degradation products can often be accelerated by for instance increasing temperature). The amount of each individual degradation product is often determined by separation of the degradation products depending on molecule size and/or charge using various chromatography techniques (e.g. SEC-HPLC and/or RP-HPLC).

Hence, as outlined above, a “stabilized formulation” refers to a formulation with increased physical stability, increased chemical stability or increased physical and chemical stability. In general, a formulation must be stable during use and storage (in compliance with recommended use and storage conditions) until the expiration date is reached.

In one embodiment of the invention the pharmaceutical formulation comprising the antibody is stable for more than 6 weeks of usage and for more than 3 years of storage.

In another embodiment of the invention the pharmaceutical formulation comprising the antibody is stable for more than 4 weeks of usage and for more than 3 years of storage.

In a further embodiment of the invention the pharmaceutical formulation comprising the antibody is stable for more than 4 weeks of usage and for more than two years of storage.

In an even further embodiment of the invention the pharmaceutical formulation comprising the antibody is stable for more than 2 weeks of usage and for more than two years of storage.

Suitable antibody formulations can also be determined by examining experiences with other already developed therapeutic monoclonal antibodies. Several monoclonal antibodies have been shown to be efficient in clinical situations, such as Rituxan (Rituximab), Herceptin (Trastuzumab) Xolair (Omalizumab), Bexxar (Tositumomab), Campath (Alemtuzumab), Zevalin, Oncolym, Humira and similar formulations may be used with the antibodies of this invention. For example, a monoclonal antibody can be supplied at a concentration of 10 mg/ml_in either 100 mg (10 ml.) or 500 mg (50 ml.) single-use vials, formulated for IV administration in 9.0 mg/ml_sodium chloride, 7.35 mg/ml_sodium citrate dihydrate, 0.7 mg/ml_polysorbate 80, and sterile water for injection. The pH is adjusted to 6.5. Alternatively, the antibody can be formulated in a solution comprising histidin, sucrose, and Polysorbate 80.

### Diagnostic Applications

The human Ficolin-3-antibodies of the invention also have non-therapeutic applications. For example, anti-human Ficolin-3 antibodies may also be useful in diagnostic assays, such as in vitro diagnostic assays for Ficolin-3 protein, e.g. detecting its expression in specific tissues, or serum. For example, anti-human Ficolin-3 antibodies could be used in assays selecting patients for anti-human Ficolin-3 treatment. For such purposes, the anti-human Ficolin-3 antibodies could be used for analyzing for the presence of human Ficolin-3 in serum or tissue specimens.

For diagnostic applications, the antibody typically will be labeled with a detectable moiety. Numerous labels are available that can be generally grouped into the following categories:

(a) Radioisotopes, such as 35S, 14C, 125I, 3H, and 131I. The antibody can be labeled with the radioisotope using the techniques described in Current Protocols in Immunology, Volumes 1 and 2, Coligen et al., Ed. Wiley-Interscience, New York, N.Y., Pubs. (1991), for example, and radioactivity can be measured using scintillation counting.
(b) Fluorescent labels such as rare-earth chelates (europium chelates) or fluorescein and its derivatives, rhodamine and its derivatives, dansyl, Lissamine, phycoerythrin and Texas Red are available. The fluorescent labels can be conjugated to the antibody using the techniques disclosed in Current Protocols in Immunology, supra, for example. Fluorescence can be quantified using a fluorimeter.
(c) Various enzyme-substrate labels are available and U.S. Pat. No. 4,275,149 provides a review of some of these. The enzyme generally catalyzes a chemical alteration of the chromogenic substrate that can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light that can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, beta-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al, “Methods for the Preparation of Enzyme-Antibody Conjugates for use in Enzyme Immunoassay,” in Methods in Enzym. (Ed., J. Langone & H. Van Vunakis), Academic Press, New York, 73:147-166 (1981).

Examples of enzyme-substrate combinations include, for example: (I) Horseradish peroxidase (HRPO) with hydrogen peroxidase as a substrate, wherein the hydrogen peroxidase oxidizes a dye precursor (e.g., orthophenylene diamine (OPD) or 3,3′,5,5′-tetramethyl benzidine hydrochloride (TMB)); (ii) alkaline phosphatase (AP) with para-nitrophenyl phosphate as chromogenic substrate; and (iii) beta-D-galactosidase (beta-D-Gal) with a chromogenic substrate (e.g., p-nitrophenyl-beta-D-galactosidase) or fluorogenic substrate 4-methylumbelliferyl-p-beta-galactosidase.

Numerous other enzyme-substrate combinations are available to those skilled in the art. For a general review of these, see U.S. Pat. No. 4,275,149 and U.S. Pat. No. 4,318,980.

Sometimes, the label is indirectly conjugated with the antibody. The skilled artisan will be aware of various techniques for achieving this. For example, the antibody can be conjugated with biotin, and any of the three broad categories of labels mentioned above can be conjugated with avidin, or vice versa. Biotin binds selectively to avidin, and thus, the label can be conjugated with the antibody in this indirect manner. Alternatively, to achieve indirect conjugation of the label with the antibody, the antibody is conjugated with a small hapten (e.g., digoxin) and one of the different types of labels mentioned above is conjugated with an anti-hapten antibody (e.g., anti-digoxin antibody). Thus, indirect conjugation of the label with the antibody can be achieved. In another embodiment of the invention, the anti-Ficolin-3 antibody need not be labeled, and the presence thereof can be detected using a labeled secondary antibody that binds to the Ficolin-3 antibody.

The antibodies of the present invention may be employed in any known assay method, such as competitive-binding assays, direct and indirect sandwich assays, and immunoprecipitation assays. Zola, Monoclonal Antibodies: A Manual of Techniques, pp. 147-158 (CRC Press, Inc. 1987).

For immunohistochemistry, the tissue sample may be fresh or frozen or may be embedded in paraffin and fixed with a preservative such as formalin, for example.

The antibodies may also be used for in vivo diagnostic assays. Generally, the antibody is labeled with a radionuclide or a non-radioactive indicator detectable by, e.g., nuclear magnetic resonance, or other means known in the art. Preferably, the label is a radiolabel, such as, e.g., 125I, 131I, 67Cu, 99 mTc, or 111In. The labeled antibody is administered to a host, preferably via the bloodstream, and the presence and location of the labeled antibody in the host is assayed. This imaging technique is suitably used in the detection, staging and treatment of neoplasms. The radioisotope is conjugated to the protein by any means, including metal-chelating compounds or lactoperoxidase, or iodogen techniques for iodination.

As a matter of convenience, the antibodies of the present invention can be provided in a kit, i.e. a packaged combination of reagents in predetermined amounts with instructions for performing the diagnostic assay. Where the antibody is labeled with an enzyme, the kit will include substrates and cofactors required by the enzyme (e.g., a substrate precursor that provides the detectable chromophore or fluorophore). In addition, other additives may be included such as stabilizers, buffers (e.g., a block buffer or lysis buffer) and the like. The relative amounts of the various reagents may be varied widely to provide for concentrations in solution of the reagents that substantially optimize the sensitivity of the assay. Particularly, the reagents may be provided as dry powders, usually lyophilized, including excipients that on dissolution will provide a reagent solution having the appropriate concentration.

### Therapeutic Applications

Methods of treating a patient using a human or humanized anti-human Ficolin-3 antibody as described herein are also provided for by the present invention. In one embodiment, the invention provides for the use of a human or humanized antibody as described herein in the preparation of a pharmaceutical composition for administration to a human patient. Typically, the patient suffers from, or is at risk for an indication associated with inflammation, apoptosis and/or autoimmunity.

Accordingly, in some embodiments the anti-human Ficolin-3 antibody according to the present invention is for the treatment of any indications associated with inflammation, apoptosis and/or autoimmunity.

In some embodiments the anti-human Ficolin-3 antibody according to the present invention is for the treatment of any autoimmune conditions such as Addison's disease, autoimmune hemolytic anemia, autoimmune thyroiditis, Crohn's disease, Graves' disease, Guillain-Barre syndrome, systemic lupus erythematosus (SLE), lupus nephritis, multiple sclerosis, myasthenia gravis, psoriasis, primary biliary cirrhosis, rheumatoid arthritis and uveitis, asthma, atherosclerosis, Type I diabetes, psoriasis, various allergies.

In some embodiments the anti-human Ficolin-3 antibody according to the present invention is for the treatment of any inflammatory disorder or condition selected from the group consisting of preeclampsia, appendicitis, peptic ulcer, gastric ulcer, duodenal ulcer, peritonitis, pancreatitis, ulcerative colitis, pseudomembranous colitis, acute colitis, ischemic colitis, diverticulitis, epiglottitis, achalasia, cholangitis, cholecystitis, hepatitis, Crohn's disease, enteritis, Whipple's disease, allergy, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, pneumonitis, pneumotransmicroscopicsilicovolcanoconiosis, alvealitis, bronchiolitis, pharyngitis, pleurisy, sinusitis, influenza, respiratory syncytial virus infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, sunburn, urticaria, warts, wheals, vasulitis, angiitis, endocarditis, arteritis, atherosclerosis, thrombophlebitis, pericarditis, myocarditis, myocardial ischemia, periarteritis nodosa, rheumatic fever, Alzheimer's disease, coeliac disease, congestive heart failure, adult respiratory distress syndrome, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thyroiditis, systemic lupus erythematosis, Goodpasture's syndrome, glomerelonephiritis, Behcet's syndrome, allograft rejection, graft-versus-host disease, Type I diabetes, ankylosing spondylitis, Berger's disease, Reiter's syndrome and Hodgkin's disease, keratitis, Type 2 diabetes, cystic fibrosis, myocardial infarction, reperfusion injury, stroke, dermatomyositis, metabolic syndrome, systemic inflammatory response syndrome, sepsis, multiple organ failure, disseminated intravascular coagulation, anaphylactic shock. Vascular complication and nephropathy associated with type 1 and/or type 2 diabetes, meningitis, bacterial septicaemia, complicated malaria, atypical haemolytic uremic syndrome, haemolytic uremic syndrome, age related macular degeneration, paroxysmal nocturnal hemoglobinuria, snake venom bite, burn injury, nephropathy, such as diabetic nephropathy and complications to organ transplantations.

In some embodiments the anti-human Ficolin-3 antibody according to the present invention is for the treatment of any inflammatory disorder selected from the group consisting of organ ischemia, reperfusion injury, organ necrosis, vasulitis, endocarditis, atherosclerosis, thrombophlebitis, pericarditis, myocarditis, myocardial ischemia, periarteritis nodosa, rheumatic fever, congestive heart failure, chronic heart failure, preeclampsia, adult respiratory distress syndrome, cerebral infarction, cerebral embolism. Vascular complications and nephropathy associated with type 1 and/or type 2 diabetes.

In some embodiments the anti-human Ficolin-3 antibody according to the present invention is for the treatment of any indications associated with coagulation, thrombotic or coagulopathic related diseases.

In some embodiments the anti-human Ficolin-3 antibody according to the present invention is used in connection with the implantation of embryonic cells or gene targeted cells. In some embodiments the anti-human Ficolin-3 antibody according to the present invention is used in connection with any type of organ or cell transplantation.

In some embodiments the anti-human Ficolin-3 antibody according to the present invention is for the treatment of an indication associated with coagulation, thrombotic or coagulopathic related diseases or disorders including inflammatory response and chronic thromboembolic diseases or disorders associated with fibrin formation including vascular disorders such as thrombosis, such as deep venous thrombosis, arterial thrombosis, post surgical thrombosis, coronary artery bypass graft (CABG), percutaneous transdermal coronary angioplastry (PTCA), platelet deposition stroke, tumor growth, tumor metastasis, angiogenesis, thrombolysis, atherosclerosis, restenosis, such as arteriosclerosis and/or restenosis following angioplastry, acute and chronic indications such as inflammation, sepsis, septic chock, septicemia, hypotension, adult respiratory distress syndrome (ARDS), systemic inflammatory response syndrome (SIRS), disseminated intravascular coagulopathy (DIC), pulmonary embolism, pathological platelet deposition, myocardial infarction, or the prophylactic treatment of mammals with atherosclerotic vessels at risk for thrombosis, venoocclusive disease following peripheral blood progenitor cell (PBPC) transplantation, hemolytic uremic syndrome (HUS), and thrombotic thrombocytopenic purpura (TTP) and rheumatic fever.

In some embodiments the anti-human Ficolin-3 antibody according to the present invention is for preventing the occurrence of thromboembolic complications in identified high risk patients, such as those undergoing surgery or those with congestive heart failure.

In some embodiments the anti-human Ficolin-3 antibody according to the present invention is for the treatment of a medical condition associated with the heart.

In some embodiments the anti-human Ficolin-3 antibody according to the present invention is for the treatment of a medical condition associated with a deficiency or an abundance or abnormal level in quantity of a polypeptide associated with complement activation.

### Dosages

For administration of the antibody, the dosage ranges from about 0.0001 to 100 mg/kg, and more usually 0.01 to 5 mg/kg, of the host body weight. For example, dosages can be about 0.3 mg/kg body weight, about 1 mg/kg body weight, about 3 mg/kg body weight, about 5 mg/kg body weight or about 10 mg/kg body weight or within the range of 1-10 mg/kg. An exemplary treatment regime entails administration twice per week, once per week, once every two weeks, once every three weeks, once every four weeks, once a month, once every 3 months or once every three to 6 months. Preferred dosage regimens for an anti-human Ficolin-3 antibody of the invention include about 1, 3, or 10 mg/kg body weight body weight via intravenous administration or subcutaneous injection, with the antibody being given using one of the following dosing schedules: (i) loading doses every 1-3 weeks for 2-4 dosages, then every two; months (ii) every four weeks; (iii) every week, or any other optimal dosing. In some methods, two or more monoclonal antibodies with different binding specificities are administered simultaneously, in which case the dosage of each antibody administered falls within the ranges indicated. Antibody is usually administered on multiple occasions. Intervals between single dosages can be, for example, weekly, monthly, every three months or yearly. Intervals can also be irregular as indicated by measuring blood levels of antibody to the target antigen in the patient. In some methods, dosage is adjusted to achieve a plasma antibody concentration of about 1-1000 μg/ml and in some methods about 25-300 μg/ml. Alternatively, antibody can be administered as a sustained release formulation, in which case less frequent administration is required. Dosage and frequency vary depending on the half-life of the antibody in the patient. In general, human antibodies show the longest half-life, followed by humanized antibodies, chimeric antibodies, and nonhuman antibodies. The dosage and frequency of administration can vary depending on whether the treatment is prophylactic or non-prophylactic (e.g., palliative or curative). In prophylactic applications, a relatively low dosage is administered at relatively infrequent intervals over a long period of time. Some patients continue to receive treatment for the rest of their lives. In palliative or curative applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

### Articles of Manufacture

In another embodiment of the invention, an article of manufacture containing materials useful for the treatment of the disorders described above is provided. For example, the article of manufacture can comprise a container containing a human or humanized anti-human Ficolin-3 antibody as described herein together with instructions directing a user to treat a disorder such as an autoimmune or inflammatory disease or disorder in a human with the antibody in an effective amount. The article of manufacture typically comprises a container and a label or package insert on or associated with the container. Suitable containers include, for example, bottles, vials, syringes, etc. The containers may be formed from a variety of materials such as glass or plastic. The container holds a composition that is effective for treating the condition and may have a sterile access port (for example, the container may be an intravenous solution bag or a vial having a stopper pierceable by a hypodermic injection needle). At least one active agent in the composition is the human or humanized anti-human Ficolin-3 antibody herein, or an antigen-binding fragment or antibody derivative (e.g., an immunoconjugate) comprising such an antibody. The label or package insert indicates that the composition is used for treating the condition of choice, such as, e.g an indication associated with inflammation, apoptosis and/or autoimmunity.

Moreover, the article of manufacture may comprise (a) a first container with a composition contained therein, wherein the composition comprises the human or humanized antibody herein, and (b) a second container with a composition contained therein, wherein the composition comprises a therapeutic agent other than the human or humanized antibody. The article of manufacture in this embodiment of the invention may further comprise a package insert indicating that the first and second compositions can be used in combination to treat an autoimmune or inflammatory disease or disorder. Such therapeutic agents may be any of the adjunct therapies described in the preceding section. Alternatively, or additionally, the article of manufacture may further comprise a second (or third) container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

SEQ ID NO: 1: Human Ficolin-3
(299 amino acid complete sequence of isoform 1, underlined
sequence is absent in the 288 amino acid sequence of isoform 2):
MDLLWILPSLWLLLLGGPACLKTQEHPSCPGPRELEASKVVLLPSCPGAPGSPGEKGAPGPQGPPGPPGK
MGPKGEPGDPVNLLRCQEGPRNCRELLSQGATLSGWYHLCLPEGRALPVFCDMDTEGGGWLVFQRRQD
GSVDFFRSWSSYRAGFGNQESEFWLGNENLHQLTLQGNWELRVELEDFNGNRTFAHYATFRLLGEVDHY
QLALGKFSEGTAGDSLSLHSGRPFTTYDADHDSSNSNCAVIVHGAWWYASCYRSNLNGRYAVSEAAAH
KYGIDWASGRGVGHPYRRVRMMLR
SEQ ID NO: 2: Human Ficolin-3
(288 amino acid complete sequence of isoform 2):
MDLLWILPSLWLLLLGGPACLKTQEHPSCPGPRELEASKVVLLPSCPGAPGSPGEKGAPGPQGPPGPPGK
MGPKGEPGPRNCRELLSQGATLSGWYHLCLPEGRALPVFCDMDTEGGGWLVFQRRQDGSVDFFRSWSS
YRAGFGNQESEFWLGNENLHQLTLQGNWELRVELEDFNGNRTFAHYATFRLLGEVDHYQLALGKFSEGT
AGDSLSLHSGRPFTTYDADHDSSNSNCAVIVHGAWWYASCYRSNLNGRYAVSEAAAHKYGIDWASGRG
VGHPYRRVRMMLR
SEQ ID NO: 3: Homo sapiens mRNA for Hakata antigen,
complete cds.
AGCAAGATGGATCTACTGTGGATCCTGCCCTCCCTGTGGCTTCTCCTGCTTGGGGGGCCTGCCTGCCT
GAAGACCCAGGAACACCCCAGCTGCCCAGGACCCAGGGAACTGGAAGCCAGCAAAGTTGTCCTCCTG
CCCAGTTGTCCCGGAGCTCCAGGAAGTCCTGGGGAGAAGGGAGCCCCAGGTCCTCAAGGGCCACCTG
GACCACCAGGCAAGATGGGCCCCAAGGGTGAGCCAGGAGATCCAGTGAACCTGCTCCGGTGCCAGGA
AGGCCCCAGAAACTGCCGGGAGCTGTTGAGCCAGGGCGCCACCTTGAGCGGCTGGTACCATCTGTGC
CTACCTGAGGGCAGGGCCCTCCCAGTCTTTTGTGACATGGACACCGAGGGGGGCGGCTGGCTGGTGT
TTCAGAGGCGCCAGGATGGTTCTGTGGATTTCTTCCGCTCTTGGTCCTCCTACAGAGCAGGTTTTGGGA
ACCAAGAGTCTGAATTCTGGCTGGGAAATGAGAATTTGCACCAGCTTACTCTCCAGGGTAACTGGGAG
CTGCGGGTAGAGCTGGAAGACTTTAATGGTAACCGTACTTTCGCCCACTATGCGACCTTCCGCCTCCTC
GGTGAGGTAGACCACTACCAGCTGGCACTGGGCAAGTTCTCAGAGGGCACTGCAGGGGATTCCCTGA
GCCTCCACAGTGGGAGGCCCTTTACCACCTATGACGCTGACCACGATTCAAGCAACAGCAACTGTGCA
GTGATTGTCCACGGTGCCTGGTGGTATGCATCCTGTTACCGATCAAATCTCAATGGTCGCTATGCAGTG
TCTGAGGCTGCCGCCCACAAATATGGCATTGACTGGGCCTCAGGCCGTGGTGTGGGCCACCCCTACC
GCAGGGTTCGGATGATGCTTCGATAGGGCACTCTGGCAGCCAGTGCCCTTATCTCTCCTGTACAGCTT
CCGGATCGTCAGCCACCTTGCCTTTGCCAACCACCTCTGCTTGCCTGTCCACATTTAAAAATAAAATCAT
TTTAGCCCTTTCAAAAAAAAAAAAAAAAAAAAAAAAAAAAA

### Specific Embodiments of the Invention

In some specific embodiments the antibody or antigen-binding fragment according to the present invention binds an epitope within Ficolin-3, which epitope is not directly associated with natural ligand binding, such as an epitope comprising the amino acid at position 166E in human Ficolin-3.

In some specific embodiments the antibody or antigen-binding fragment according to the present invention binds within a domain directly associated with natural ligand binding, such as in the S1 domain.

In some specific embodiments the antibody or antigen-binding fragment according to the present invention is an antibody, wherein, upon binding to human Ficolin-3, there is a 60% to 90% reduction in the ability of Ficolin-3 to bind to its natural ligand.

In some specific embodiments the antibody or antigen-binding fragment according to the present invention is an antibody, wherein, upon binding to human Ficolin-3, there is a 60% to 90% reduction in deposition of complement factor C4.

In some specific embodiments the antibody or antigen-binding fragment according to the present invention is an antibody, which inhibit rFicolin-3 binding to acetyled bovine serum albumin (BSA).

In some specific embodiments the antibody or antigen-binding fragment according to the present invention is an antibody, which inhibits complement activation as measured by C4, C3 and/or TCC deposition on acetylated BSA.

In some specific embodiments the antibody or antigen-binding fragment according to the present invention is an antibody, which binds to human Ficolin-3 with a KD of 10 nM or less, e.g. 5 nM or less, such as 2 nM or less, e.g. 1 nM or less, as determined in the Biocore assay described herein.

In some specific embodiments the antibody or antigen-binding fragment according to the present invention is an antibody, which competes with a reference antibody in binding to human Ficolin-3, wherein the reference antibody comprises:

a heavy-chain variable region comprising the sequence corresponding to the sequence in any one of anti-Ficolin-3 antibody FCN308, FCN334, or FCN329 and a light-chain variable region comprising the sequence corresponding to the sequence in any one of anti-Ficolin-3 antibody FCN308, FCN334, or FCN329, each of clone FCN308, FCN334, or FCN329 deposited under Ref. No.: Q9190.

Deposits of hybridoma cells to HPACC (Ref. No.: Q9190):

Hybridoma cells contain: 12 Vials of PG-HYB-308, 12 Vials of PG-HYB-329, 12 Vials of PG-HYB-334, and 3×100 mls of culture medium for testing was deposited at Health Protection Agency Culture Collections (HPACC), Microbiology Services Division, Porton Down, Salisbury Wiltshire, SP4 0JG. UK, Ref. No.: Q9190:

| Cell Line | Accession Number |
|---|---|
| Hybridoma PG-HYB-FCN329 | 11062401 |
| Hybridoma PG-HYB-FCN334 | 11062402 |
| Hybridoma PG-HYB-FCN308 | 11062403 |

In some specific embodiments the antibody or antigen-binding fragment according to the present invention is an antibody, which competes with anti-Ficolin-3 antibody FCN308, FCN334, or FCN329 in binding to human Ficolin-3, each of clone FCN308, FCN334, or FCN329 deposited under Ref. No.: Q9190.

In some specific embodiments the antibody or antigen-binding fragment according to the present invention comprises a nucleotide sequence encoding the constant region of a light chain, a heavy chain or both light and heavy chains of a human antibody.

In some specific embodiments the antibody or antigen-binding fragment according to the present invention is human or humanized.

In some specific embodiments the antibody or antigen-binding fragment according to the present invention is a full-length antibody, such as an IgG4 antibody.

In some specific embodiments the antibody or antigen-binding fragment according to the present invention is an antibody fragment or a single-chain antibody, such as a single-chain variable fragment (scFv).

In some specific embodiments the antibody or antigen-binding fragment according to the present invention is conjugated to another moiety, such as a cytotoxic moiety, a radioisotope or a drug.

In some specific embodiments the antibody or antigen-binding fragment according to the present invention binds to an epitope comprising amino acids at a position selected from 166E, 237D, 239D, 241S, 243S, 258C, 259Y, 277Y, 287V of SEQ ID NO:1, or any combination thereof.

In some specific embodiments the antibody or antigen-binding fragment according to the present invention competitively inhibits binding of any one antibody selected from the antibodies deposited under Ref. No.: Q9190 to human ficolin-3.

In some specific embodiments the antibody or antigen-binding fragment according to the present invention is an antibody, wherein, when administered to a human patient via intravenous infusion, the antibody provides complete complement inhibition at dosages below 0.005 g/kg.

In some specific embodiments the antibody or antigen-binding fragment according to the present invention is an antibody, wherein, when administered to a human patient via intravenous infusion, the antibody provides therapeutic benefits at dosages below 0.002 g/kg.

In a further aspect the present invention relates to a method for inhibiting complement activation in a subject in need thereof the method comprising administering an antibody or antigen-binding fragment according to the invention to a human subject in need thereof.

In some specific embodiments the subject is suffering from or at risk for such an indication or condition selected from a condition such as Addison's disease, autoimmune hemolytic anemia, autoimmune thyroiditis, Crohn's disease, Graves' disease, Guillain-Barre syndrome, systemic lupus erythematosus (SLE), lupus nephritis, multiple sclerosis, myasthenia gravis, psoriasis, primary biliary cirrhosis, rheumatoid arthritis and uveltis, asthma, atherosclerosis, Type I diabetes, psoriasis, various allergies.

In some specific embodiments the subject is suffering from or at risk for such an indication or condition selected from the group consisting of preeclampsia, appendicitis, peptic ulcer, gastric ulcer, duodenal ulcer, peritonitis, pancreatitis, ulcerative colitis, pseudomembranous colitis, acute colitis, ischemic colitis, diverticulitis, epiglottitis, achalasia, cholangitis, cholecystitis, hepatitis, Crohn's disease, enteritis, Whipple's disease, allergy, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, pneumonitis, pneumotransmicroscopicsilicovolcanoconiosis, alvealitis, bronchiolitis, pharyngitis, pleurisy, sinusitis, influenza, respiratory syncytial virus infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, sunburn, urticaria, warts, wheals, vasulitis, angiitis, endocarditis, arteritis, atherosclerosis, thrombophlebitis, pericarditis, myocarditis, myocardial ischemia, periarteritis nodosa, rheumatic fever, Alzheimer's disease, coeliac disease, congestive heart failure, adult respiratory distress syndrome, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciltis, Paget's disease, gout, periodontal disease, rheumatoid arthritis, synovitis, myasthenia gravis, thyroiditis, systemic lupus erythematosis, Goodpasture's syndrome, Behcet's syndrome, allograft rejection, graft-versus-host disease, Type I diabetes, ankylosing spondylitis, Berger's disease, Reiter's syndrome and Hodgkin's disease, keratitis, Type 2 diabetes, cystic fibrosis, myocardial infarction, reperfusion injury, stroke, dermatomyositis, metabolic syndrome, systemic inflammatory response syndrome, sepsis, multiple organ failure, disseminated intravascular coagulation, anaphylactic shock. Vascular complication and nephropathy associated with type 1 and/or type 2 diabetes, meningitis, bacterial septicaemia, complicated malaria, atypic haemolytic uremic syndrome, haemolytic uremic syndrome, age related macular degeneration, paroxysmal nocturnal hemoglobinuria, snake venom bite, burn injury, nephropathy, such as diabetic nephropathy and complications to organ transplantations.

In some specific embodiments the subject is suffering from or at risk for such an indication or condition selected from the group consisting of organ ischemia, reperfusion injury, organ necrosis, vasulitis, endocarditis, atherosclerosis, thrombophlebitis, pericarditis, myocarditis, myocardial ischemia, periarteritis nodosa, rheumatic fever, congestive heart failure, adult respiratory distress syndrome, cerebral infarction, cerebral embolism. Vascular complications and nephropathy associated with type 1 and/or type 2 diabetes.

In some specific embodiments the subject is suffering from or at risk for such an indication or condition selected from any indications associated with coagulation, thrombotic or coagulopathic related diseases, thrombotic or coagulopathic related diseases or disorders including inflammatory response and chronic thromboembolic diseases or disorders associated with fibrin formation including vascular disorders such as thrombosis, such as deep venous thrombosis, arterial thrombosis, post-surgical thrombosis, coronary artery bypass graft (CABG), percutaneous transdermal coronary angioplastry (PTCA), platelet deposition stroke, tumor growth, tumor metastasis, angiogenesis, thrombolysis, atherosclerosis, restenosis, such as arteriosclerosis and/or restenosis following angioplastry, acute and chronic indications such as inflammation, sepsis, septic shock, septicemia, hypotension, adult respiratory distress syndrome (ARDS), systemic inflammatory response syndrome (SIRS), disseminated intravascular coagulopathy (DIC), pulmonary embolism, pathological platelet deposition, myocardial infarction, or the prophylactic treatment of mammals with atherosclerotic vessels at risk for thrombosis, venoocclusive disease following peripheral blood progenitor cell (PBPC) transplantation, hemolytic uremic syndrome (HUS), and thrombotic thrombocytopenic purpura (TTP) and rheumatic fever.

In some specific embodiments the method for inhibiting complement activation in a subject in need thereof is for preventing the occurrence of thromboembolic complications in identified high risk patients, such as those undergoing surgery or those with congestive heart failure.

In some specific embodiments the method for inhibiting complement activation in a subject in need thereof is for the treatment of a medical condition associated with the heart.

In some specific embodiments the method for inhibiting complement activation in a subject in need thereof is for the treatment of cancer, such as a hematological cancer.

In some specific embodiments the method for inhibiting complement activation in a subject in need thereof is for the treatment of a medical condition associated with a deficiency or an abundance or abnormal level in quantity of a polypeptide associated with complement activation, such as Ficolin-3.

### NUMBERED EMBODIMENTS OF THE INVENTION

### Embodiment 1

An isolated recombinant human monoclonal antibody, or an antigen-binding fragment thereof, which exhibit specific binding to human Ficolin-3 and which inhibits complement activation in a human body fluid.

### Embodiment 2

The antibody or antigen-binding fragment according to embodiment 1, which antibody binds an epitope within Ficolin-3, which epitope is not directly associated with natural ligand binding, such as an epitope comprising the amino acid at position 166E in human Ficolin-3.

### Embodiment 3

The antibody or antigen-binding fragment according to any one of embodiments 1 or 2, which antibody binds within a domain directly associated with natural ligand binding, such as in the S1 domain.

### Embodiment 4

The antibody or antigen-binding fragment according to any one of embodiments 1-3, wherein, upon binding to human Ficolin-3, there is a 60% to 90% reduction in the ability of Ficolin-3 to bind to its natural ligand.

### Embodiment 5

The antibody or antigen-binding fragment according to any one of embodiments 1-4, wherein, upon binding to human Ficolin-3, there is a 60% to 90% reduction in deposition of complement factor C4.

### Embodiment 6

The antibody or antigen-binding fragment according to any one of embodiments 1-5, which inhibit rFicolin-3 binding to acetyled bovine serum albumin (BSA).

### Embodiment 7

The antibody or antigen-binding fragment according to any one of embodiments 1-6, which inhibits complement activation as measured by C4, C3 and/or TCC deposition on acetylated BSA.

### Embodiment 8

The antibody or antigen-binding fragment according to any one of embodiments 1-7, which binds to human Ficolin-3 with a KD of 10 nM or less, e.g. 5 nM or less, such as 2 nM or less, e.g. 1 nM or less, as determined in the Biocore assay described herein.

### Embodiment 9

The antibody or antigen-binding fragment according to any of the preceding embodiments, which competes with a reference antibody in binding to human Ficolin-3, wherein the reference antibody comprises:
a heavy-chain variable region comprising the sequence corresponding to the sequence in any one of anti-Ficolin-3 antibody FCN308, FCN334, or FCN329 and a light-chain variable region comprising the sequence corresponding to the sequence in any one of anti-Ficolin-3 antibody FCN308, FCN334, or FCN329, each of clone FCN308, FCN334, or FCN329 deposited under Ref. No.: Q9190.

### Embodiment 10

The antibody or antigen-binding fragment according to any of the preceding embodiments, which competes with anti-Ficolin-3 antibody FCN308, FCN334, or FCN329 in binding to human Ficolin-3, each of clone FCN308, FCN334, or FCN329 deposited under Ref. No.: Q9190.

### Embodiment 11

The antibody or antigen-binding fragment according to any of the preceding embodiments, comprising a nucleotide sequence encoding the constant region of a light chain, a heavy chain or both light and heavy chains of a human antibody.

### Embodiment 12

The antibody or antigen-binding fragment according to any of the preceding embodiments, which is human or humanized.

### Embodiment 13

The antibody or antigen-binding fragment according to any of the preceding embodiments, wherein the antibody is a full-length antibody, such as an IgG4 antibody.

### Embodiment 14

The antibody or antigen-binding fragment according to any of the preceding embodiments, wherein the antibody is an antibody fragment or a single-chain antibody, such as a single-chain variable fragment (scFv).

### Embodiment 15

The antibody or antigen-binding fragment according to any of the preceding embodiments, which antibody is conjugated to another moiety, such as a cytotoxic moiety, a radioisotope or a drug.

### Embodiment 16

The antibody or antigen-binding fragment according to any of the preceding embodiments, which binds to an epitope comprising amino acids at a position selected from 166E, 237D, 239D, 241S, 243S, 258C, 259Y, 277Y, 287V of SEQ ID NO:1, or any combination thereof.

### Embodiment 17

The antibody or antigen-binding fragment according to any of the preceding embodiments, wherein said antibody or antigen binding fragment competitively inhibits binding of any one antibody selected from the antibodies to human ficolin-3 deposited at HPACC under Provisional Accession Numbers 11062401, 11062402, or 11062403.

### Embodiment 18

The antibody or antigen-binding fragment according to any of the preceding embodiments, wherein, when administered to a human patient via intravenous infusion, the antibody provides complete complement inhibition at dosages below 0.005 g/kg.

### Embodiment 19

The antibody or antigen-binding fragment according to any of the preceding embodiments, wherein, when administered to a human patient via intravenous infusion, the antibody provides therapeutic benefits at dosages below 0.002 g/kg.

### Embodiment 20

A composition comprising an antibody as defined in any of embodiments 1-19.

### Embodiment 21

An expression vector comprising a nucleotide sequence encoding an antibody as defined in any of embodiments 1-19.

### Embodiment 22

A recombinant eukaryotic or prokaryotic host cell which produces an antibody as defined in any of embodiments 1 to 19.

### Embodiment 23

A hybridoma which produces an antibody as defined in any of embodiments 1 to 19.

### Embodiment 24

A method of producing an anti-Ficolin-3 antibody, or an antigen-binding fragment thereof according to any one of embodiments 1-19, comprising culturing a host cell comprising a nucleic acid encoding said antibody under suitable conditions and recovering said antibody or antigen-binding fragment thereof.

### Embodiment 25

Use of an antibody or antigen-binding fragment of any of embodiments 1-19 or other ficolin-3 inhibitor for the inhibition of ficolin-3 recognition to its natural ligands.

### Embodiment 26

A method for treating an indication or condition associated with ficolin-3 natural ligand recognition, such as an indication associated with inflammation, coagulation, apoptosis and/or autoimmunity comprising administering the antibody or antigen-binding fragment of any of embodiments 1-19 or other ficolin-3 inhibitor to a human subject suffering from or at risk for such an indication or condition.

### Embodiment 27

A method for inhibiting complement activation in a subject in need thereof the method comprising administering an antibody or antigen-binding fragment of any of embodiments 1-19 or other ficolin-3 inhibitor to a human subject in need thereof.

### Embodiment 28

The method of embodiments 26 or 27, wherein the subject is suffering from or at risk for such an indication or condition selected from a condition such as Addison's disease, autoimmune hemolytic anemia, autoimmune thyroiditis, Crohn's disease, Graves' disease, Guillain-Barre syndrome, systemic lupus erythematosus (SLE), lupus nephritis, multiple sclerosis, myasthenia gravis, psoriasis, primary biliary cirrhosis, rheumatoid arthritis and uveitis, asthma, atherosclerosis, Type I diabetes, psoriasis, various allergies.

### Embodiment 29

The method of embodiments 26 or 27, wherein the subject is suffering from or at risk for such an indication or condition selected from the group consisting of preeclampsia, appendicitis, peptic ulcer, gastric ulcer, duodenal ulcer, peritonitis, pancreatitis, ulcerative colitis, pseudomembranous colitis, acute colitis, ischemic colitis, diverticulitis, epiglottitis, achalasia, cholangitis, cholecystitis, hepatitis, Crohn's disease, enteritis, Whipple's disease, allergy, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, pneumonitis, pneumotransmicroscopicsilicovolcanoconiosis, alvealitis, bronchiolitis, pharyngitis, pleurisy, sinusitis, influenza, respiratory syncytial virus infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, sunburn, urticaria, warts, wheals, vasulitis, angiitis, endocarditis, arteritis, atherosclerosis, thrombophlebitis, pericarditis, myocarditis, myocardial ischemia, periarteritis nodosa, rheumatic fever, Alzheimer's disease, coeliac disease, congestive heart failure, adult respiratory distress syndrome, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thyroiditis, systemic lupus erythematosis, Goodpasture's syndrome, Behcet's syndrome, allograft rejection, graft-versus-host disease, Type I diabetes, ankylosing spondylitis, Berger's disease, Reiter's syndrome and Hodgkin's disease, keratitis, Type 2 diabetes, cystic fibrosis, myocardial Infarction, reperfusion injury, stroke, dermatomyositis, metabolic syndrome, systemic Inflammatory response syndrome, sepsis, multiple organ failure, disseminated intravascular coagulation, anaphylactic shock. Vascular complication and nephropathy associated with type 1 and/or type 2 diabetes, meningitis, bacterial septicaemia, complicated malaria, atypic haemolytic uremic syndrome, haemolytic uremic syndrome, age related macular degeneration, paroxysmal nocturnal hemoglobinuria, snake venom bite, burn injury, nephropathy, such as diabetic nephropathy and complications to organ transplantations.

### Embodiment 30

The method of embodiments 26 or 27, wherein the subject is suffering from or at risk for such an indication or condition selected from the group consisting of organ Ischemia, reperfusion injury, organ necrosis, vasulitis, endocarditis, atherosclerosis, thrombophlebitis, pericarditis, myocarditis, myocardial ischemia, periarteritis nodosa, rheumatic fever, congestive heart failure, adult respiratory distress syndrome, cerebral infarction, cerebral embolism. Vascular complications and nephropathy associated with type 1 and/or type 2 diabetes.

### Embodiment 31

The method of embodiments 26 or 27, wherein the subject is suffering from or at risk for such an indication or condition selected from any indications associated with coagulation, thrombotic or coagulopathic related diseases, thrombotic or coagulopathic related diseases or disorders including inflammatory response and chronic thromboembolic diseases or disorders associated with fibrin formation including vascular disorders such as thrombosis, such as deep venous thrombosis, arterial thrombosis, post surgical thrombosis, coronary artery bypass graft (CABG), percutaneous transdermal coronary angioplastry (PTCA), platelet deposition stroke, tumor growth, tumor metastasis, angiogenesis, thrombolysis, atherosclerosis, restenosis, such as arteriosclerosis and/or restenosis following angioplastry, acute and chronic indications such as inflammation, sepsis, septic chock, septicemia, hypotension, adult respiratory distress syndrome (ARDS), systemic inflammatory response syndrome (SIRS), disseminated intravascular coagulopathy (DIC), pulmonary embolism, pathological platelet deposition, myocardial infarction, or the prophylactic treatment of mammals with atherosclerotic vessels at risk for thrombosis, venoocclusive disease following peripheral blood progenitor cell (PBPC) transplantation, hemolytic uremic syndrome (HUS), and thrombotic thrombocytopenic purpura (TTP) and rheumatic fever.

### Embodiment 32

The method of embodiments 26 or 27, for preventing the occurrence of thromboembolic complications in identified high risk patients, such as those undergoing surgery or those with congestive heart failure.

### Embodiment 33

The method of embodiments 26 or 27, for the treatment of a medical condition associated with the heart.

### Embodiment 34

The method of embodiments 26 or 27, for the treatment of cancer, such as a hematological cancer.

### Embodiment 35

The method of embodiments 26 or 27, for the treatment of a medical condition associated with a deficiency or an abundance or abnormal level in quantity of a polypeptide associated with complement activation, such as Ficolin-3.

### Embodiment 36

A pharmaceutical composition comprising an antibody as defined in any of embodiments 1 to 19, or other ficolin-3 inhibitor, and a pharmaceutically acceptable carrier.

### Embodiment 37

The antibody as defined in any of embodiments 1 to 19, or other ficolin-3 inhibitor for use as a medicament.

### Embodiment 38

The antibody or other ficolin-3 inhibitor according to embodiment 37, wherein the use is for treatment of an indication, condition or disease as defined in any one of embodiments 27-35.

### Embodiment 39

A diagnostic composition comprising an antibody as defined in any of embodiments 1 to 19, or other ficolin-3 inhibitor.

### Embodiment 40

A method for detecting the presence of human Ficolin-3 in a sample, the method comprising the steps of:
a) contacting the sample with an anti-Ficolin-3 antibody of any of embodiments 1 to 19, or other ficolin-3 inhibitor under conditions that allow for formation of a complex between the antibody and human Ficolin-3; and
b) analyzing whether a complex has been formed.

### Embodiment 41

A kit for detecting the presence of human Ficolin-3 in a sample comprising
a) an anti-Ficolin-3 antibody of any of embodiments 1 to 19, or other ficolin-3 inhibitor; and
b) instructions for use of the kit.

### EXAMPLES

### Example 1

In order to learn more of their properties the panel of monoclonal mouse antibodies raised against Ficolin-3 was investigated in different enzyme-linked immunosorbent assays (ELISAs).

Antibody Reactivity Against Ficolin-3—

Supernatants containing monoclonal mouse-anti-Ficolin-3 antibodies (FCN3) were collected from hybridoma clones. In order to test the reactivity against Ficolin-3, a simple ELISA setup was used wherein rFicolin-3 (produced in-house) was coated directly in a microtiter plate [1 μg/ml] in phosphate buffered saline (PBS) over night (ON) 4° C. The plate was washed thrice in barbital buffer (4 mM C₈H₁₁N₂NaO₃, 145 mM NaCl, 2.6 mM CaCl₂, 2.1 mM MgCl₂, pH=7.4) containing 0.05% Tween 20 (VBS-T). The FCN3 antibody supernatants were added on to the rFicolin-3 in a 2-fold serial dilution made in VBS-T and incubated shaking for 2 hours room temperature (RT). After washing as above, the antibody binding was detected with rabbit-anti-mouse-HRP (from DAKO) [1:2000] for 1 hour RT. The plate was washed and developed using OPD substrate containing H₂O₂. Optical density was measured at 490 nm.

Inhibition of Ficolin-3 Binding to Ligand in ELISA—

Next step was to investigate whether any of the Fcolin-3 antibodies had epitopes in the fibrinogen-like domain of the Ficolin molecule and thereby would interfere with the binding capacity of the protein. As ligand for Ficolin-3 in ELISA acBSA was coated to microtiter wells [5 μg/ml] in PBS ON at 4° C. Next, rFicolin-3 [2 μg/ml] or serum [1:50] and the different FCN3 antibodies in 2-fold serial dilution preincubated at 4° C. for 30 min. An isotype matched control antibody with no human specificity was added as well. Meanwhile the plates with acBSA were washed in VBS-T before the rFicolin-3/serum with FCN3 antibodies were added to the ELISA plate and incubated for 2 hours at 37° C. The plates were washed and detection of Ficolin-3 binding to acBSA was performed with either biotinylated monoclonal mouse-anti-Ficolin-3 (FCN334) or alternatively biotinylated polyclonal antibody against Ficolin-3 (from R&D Systems) to circumvent the possible occurrence of overlapping epitopes between inhibiting and detecting antibodies. After incubation for 2 hours at RT the plates were washed again and streptavidin-HRP [1:2000](from GE Healthcare) was added for 1 hour before developing as described above.

Inhibition of Ficolin-3 Mediated Complement Deposition in ELISA—

To confirm that the ligand binding inhibiting property of the FCN3 antibody also resulted in inhibition of Ficolin-3 mediated complement activation, acBSA was again used as a ligand in the ELISA platform. Furthermore, by evaluating the downstream effects of Ficolin-3, antibodies competing with the binding area of the MASP's and thereby inhibiting complement activation could potentially also be exposed.

AcBSA was coated on to microtiter wells [5 μg/ml] in PBS ON 4° C. For the complement assay sodium polyanethole sulfonate (SPS) a known inhibitor of the classical as well as the alternative pathway, which leaves the lectin pathway intact, was used [Palarasah et al. 2010]. First, full serum preincubated with SPS [0.5 mg/ml] for 5 minutes on ice. Second, serum [1:80] preincubated with the different antibodies 30 minutes at 4° C. and was then added to the wells with acBSA in 2-fold serial dilution and incubated for 30 minutes at 37° C. C4- and C3-deposition on acBSA was detected with pAb rabbit-anti-huC4 (from DAKO) and pAb rabbit-anti-huC3 (from Dahde-Behring), respectively. The plates incubated for 2 hours RT and subsequently, donkey-anti-rabbit-HRP (from GE Healthcare) was applied for 1 hour before development as described. Between all steps plates were washed with VBS-T.

The evaluation of the terminal complement pathway was performed as above, except that the plates incubated with serum/antibodies for 45 min. at 37° C. Formation of the terminal complement complex (TCC) was detected with mouse-anti-C5b-9 (from Bioporto Diagnostics) and subsequently rabbit-anti-mouse-HRP.

The results as presented in the figures showed that clone FCN308 and clone FCN334 inhibited the binding of ficolin-3 to acBSA. Clone FCN308, clone 334 and FCN329 inhibited further downstream complement deposition evaluated as C4, C3 and TCC deposition. While this was not the case for the other anti-ficolin-3 clones tested.

### Example 2

### Association of Complement Activation and Adverse Outcome after Kidney Transplantation

The complement activation has been shown to be associated with adverse outcome after kidney transplantation. Particularly deposition of complement factor C4d has been shown to be a good marker of humoral rejection episodes. However, the mechanisms behind these episodes are only partly resolved. Thus the inventors of the present application speculated whether high serum levels of ficolin-3, which is one of the factors that initiate C4d deposition in other settings could be a contributing factor to kidney rejection.

### Patients and Methods

We have investigated whether ficolin-3 levels may determine the outcome of kidney transplantation. Ficolin-3 and complement factors C4 and C3 were measured in pre-transplant as well as in control serum samples. In the controls, deposition of ficolin-3, C4, C3 and the terminal complement complex (TCC) were measured in an assay based on acetylated albumin as matrix. In the study we included 527 patients that were receiving a kidney over a ten years period at the transplantation center in Copenhagen; 97 blood donors served as controls.

The patient graft survival were monitored after one year follow-up-Using receiver operating curve (ROC) analysis, 46 μg/ml ficolin-3 was chosen as the best cut off value to discriminate between high and low ficolin-3 values. Ficolin-3 serum levels were measured according to the method described in L. Munthe-Fog et al. Characterization of a polymorphism in the coding sequence of FCN3 resulting in a Ficolin-3 (Hakata antigen) deficiency state. Mol. Immunol. 45:2660-2666, 2008.

### Results

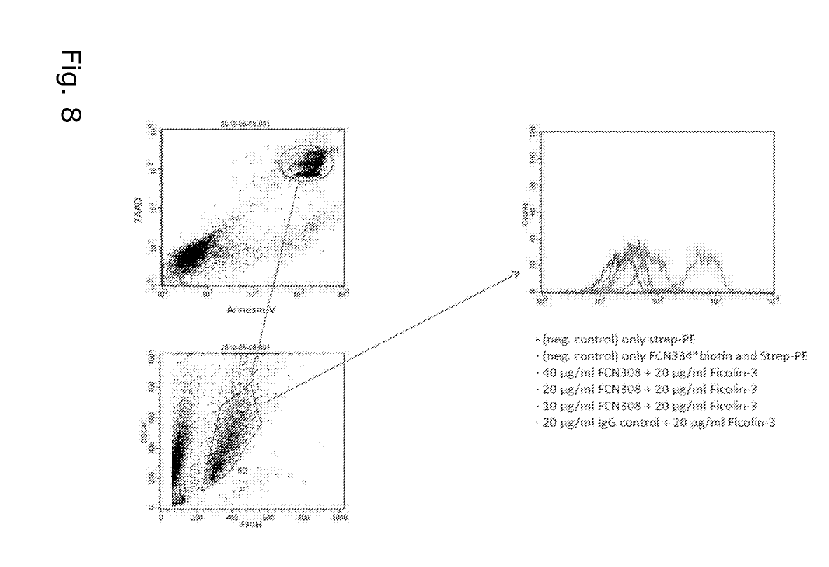

A significant increased number of patients with high ficolin-3 levels compared with those with low levels experienced loss of kidney after one year follow up (P=0.01) (FIG. 7).

It was found that concentration of ficolin-3, C4 and C3 was increased in the patients compared with the controls (p<0.001). The ficolin-3 levels correlated with the serum levels of C4 and C3 both in the patients (rho: 0.44, p<0.0001, rho: 0.53, p<0.0001, respectively) and in the controls (rho: 0.30, p<0.0038, rho: 0.50, p<0.0001, respectively). The serum levels of ficolin-3 correlated with ficolin-3 (rho 0.66, p<0.0001), C4 (rho 0.45, p<0.0001), C3 (rho 0.46, p<0.0001) and TCC (rho 0.24, p=0.0197) deposition. In a multivariable regression analysis adjusted for confounders a high ficolin-3 level was a predictor of death censored graft survival (HR=3.18, 95% CI: 1.48-6.81) (p=0.003), while this was not seen for C4 and C3.

### CONCLUSION

High levels of serum ficolin-3 is associated with decreased kidney graft survival. Thus high levels of ficolin-3 may be one of the factors that initiate kidney rejection.

The study thus verified that Ficolin-3 is involved in the pathophysiology of kidney graft rejection. The ficolin-3 levels correlated closely with the concentration and deposition of downstream serum complement components, illustrating the importance of ficolin-3 as an Initiator molecule of complement activation.

### Example 3

### Cells

Jurkat T cells (cell line E 6.1) were grown in RPMI+(RPMI-1640 supplemented with 90 U/ml penicillin, 90 μg/ml streptomycin, 2 mM L-glutamine and 10% heat-inactivated fetal calf serum (H.I. FCS)) at 37° C. and 5% CO₂ atmosphere.

### Induction of Necrosis

3×10⁶ Jurkat T cells were washed in RPMI+ and centrifuged at 1800 rpm. Cells were resuspended in TBS (10 mM Tris, 150 mM NaCl, 1.5 mM CaCl₂, pH 7.4)+1 mM MgCl₂ and rendered necrotic by incubation at 56° C. for 30 min.

Inhibition of rFicolin-3 Binding to Necrotic Jurkat T Cells

rFicolin-3 was preincubated for 30 min. at 4° C. with varying concentrations of Ficolin-3 Inhibitory antibody FCN308 or isotype antibody control. A total of 0.1×10⁶ cells/sample were washed in wash buffer (TBS+2.5 mM CaCl₂+1% H.I. FCS, pH 7.4) and centrifuged at 1800 rpm, 5 min., 4° C. The necrotic cells were incubated with rFicolin-3 and FCN308 for 1 hour at 37° C. Next, the cells were washed again and detection of rFIcolin-3 binding was performed with biotinylated monoclonal mouse-anti-ficolin-3 antibody (FCN334*biotin) for 30 min., 4° C. Cells were washed again and incubated with streptavidin-PE (BD Biosciences) for 15 min. 4° C. In this last step, necrosis was verified by staining the cells with 7-AAD and annexin V-FITC (BD Biosciences). Finally, rFicolin-3 binding to the cells and inhibition of same was analyzed by flow cytometry on FACSCalibur.

### Results

Necrotic cells were identified by 7-AAD and annexin V-FITC labeling and then gated according to the forward and side scatter properties. A concentration of 20 μg/ml rFicolin-3 in the presence of an isotype control nonsense antibody yielded a clear binding to the necrotic Jurkat T cells. The binding was dose dependently inhibited by increasing concentration of Ficolin-3 inhibitory antibody FCN308 as is apparent from FIG. 8.

This demonstrates that it is possible to inhibit binding of Ficolin-3 to necrotic/damaged cells.

1. An isolated recombinant human monoclonal antibody, or an antigen-binding fragment thereof which exhibit specific binding to human Ficolin-3 and which inhibits complement activation in a human body fluid.

2. The antibody or antigen-binding fragment according to claim 1, which antibody binds an epitope within Ficolin-3, which epitope is not directly associated with natural ligand binding, such as an epitope comprising the amino acid at position 166E in human Ficolin-3.

3. The antibody or antigen-binding fragment according to claim 1, which antibody binds within a domain directly associated with natural ligand binding, such as in the S1 domain.

4. The antibody or antigen-binding fragment according to claim 1, wherein, upon binding to human Ficolin-3, there is a 60% to 90% reduction in the ability of Ficolin-3 to bind to its natural ligand.

5. The antibody or antigen-binding fragment according to claim 1, wherein, upon binding to human Ficolin-3, there is a 60% to 90% reduction in deposition of complement factor C4.

6. The antibody or antigen-binding fragment according to claim 1, which inhibits complement activation as measured by C4, C3 and/or TCC deposition on acetylated BSA.

7. The antibody or antigen-binding fragment according to claim 1, which binds to human Ficolin-3 with a KD of 10 nM or less, e.g. 5 nM or less, such as 2 nM or less, e.g. 1 nM or less, as determined in the Biocore assay described herein.

8. The antibody or antigen-binding fragment according to claim 1, which is human or humanized.

9. The antibody or antigen-binding fragment according to claim 1, wherein the antibody is a full-length antibody, such as an IgG4 antibody.

10. The antibody or antigen-binding fragment according to claim 1, which antibody is conjugated to another moiety, such as a cytotoxic moiety, a radioisotope or a drug.

11. The antibody or antigen-binding fragment according to claim 1, which binds to an epitope comprising amino acids at a position selected from 166E, 237D, 239D, 241S, 243S, 258C, 259Y, 277Y, 287V of SEQ ID NO:1, or any combination thereof.

12. Use of an antibody or antigen-binding fragment of claim 1 or other ficolin-3 inhibitor for the inhibition of ficolin-3 recognition to its natural ligands.

13. A pharmaceutical composition comprising an antibody as defined in claim 1, or other ficolin-3 inhibitor, and a pharmaceutically acceptable carrier.

14. The antibody or other ficolin-3 inhibitor according to claim 1 for use as a medicament in the treatment of an indication, condition or disease associated with ficolin-3 natural ligand recognition, such as an indication associated with inflammation and/or coagulation and/or apoptosis, and/or allograft rejection and/or autoimmunity.

15. The method antibody or other ficolin-3 inhibitor according to claim 14, wherein the subject is suffering from or at risk for such an indication or condition selected from a condition such as Addison's disease, autoimmune hemolytic anemia, autoimmune thyroiditis, Crohn's disease, Graves' disease, Guillain-Barre syndrome, systemic lupus erythematosus (SLE), lupus nephritis, multiple sclerosis, myasthenia gravis, psoriasis, primary biliary cirrhosis, rheumatoid arthritis and uveitis, asthma, atherosclerosis, Type I diabetes, psoriasis, various allergies, preeclampsia, appendicitis, peptic ulcer, gastric ulcer, duodenal ulcer, peritonitis, pancreatitis, ulcerative colitis, pseudomembranous colitis, acute colitis, ischemic colitis, diverticulitis, epiglottitis, achalasia, cholangitis, cholecystitis, hepatitis, Crohn's disease, enteritis, Whipple's disease, allergy, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, pneumonitis, pneumotransmicroscopicsilicovolcanoconiosis, alvealitis, bronchiolitis, pharyngitis, pleurisy, sinusitis, influenza, respiratory syncytial virus infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, sunburn, urticaria, warts, wheals, vasulitis, angiitis, endocarditis, arteritis, atherosclerosis, thrombophlebitis, pericarditis, myocarditis, myocardial ischemia, periarteritis nodosa, rheumatic fever, Alzheimer's disease, coeliac disease, congestive heart failure, adult respiratory distress syndrome, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thyroiditis, systemic lupus erythematosis, Goodpasture's syndrome, Behcet's syndrome, allograft rejection, graft-versus-host disease, Type I diabetes, ankylosing spondylitis, Berger's disease, Reiter's syndrome and Hodgkin's disease, keratitis, Type 2 diabetes, cystic fibrosis, myocardial infarction, reperfusion injury, stroke, dermatomyositis, metabolic syndrome, systemic inflammatory response syndrome, sepsis, multiple organ failure, disseminated intravascular coagulation, anaphylactic shock. Vascular complication and nephropathy associated with type 1 and/or type 2 diabetes, meningitis, bacterial septicaemia, complicated malaria, atypic haemolytic uremic syndrome, haemolytic uremic syndrome, age related macular degeneration, paroxysmal nocturnal hemoglobinuria, snake venom bite, burn injury, nephropathy, such as diabetic nephropathy and complications to organ transplantations.


SEQUENCE LISTING (3 sequences)

SEQ ID NO: 1 - protein, 299 aa, Homo sapiens
MDLLWILPSLWLLLLGGPACLKTQEHPSCPGPRELEASKVVLLPSCPGAPGSPGEKGAPGPQGPPGPPGKMGPKGEPGDPVNLLRCQEGPRNCRELLSQGATLSGWYHLCLPEGRALPVFCDMDTEGGGWLVFQRRQDGSVDFFRSWSSYRAGFGNQESEFWLGNENLHQLTLQGNWELRVELEDFNGNRTFAHYATFRLLGEVDHYQLALGKFSEGTAGDSLSLHSGRPFTTYDADHDSSNSNCAVIVHGAWWYASCYRSNLNGRYAVSEAAAHKYGIDWASGRGVGHPYRRVRMMLR

SEQ ID NO: 2 - protein, 288 aa, Homo sapiens
MDLLWILPSLWLLLLGGPACLKTQEHPSCPGPRELEASKVVLLPSCPGAPGSPGEKGAPGPQGPPGPPGKMGPKGEPGPRNCRELLSQGATLSGWYHLCLPEGRALPVFCDMDTEGGGWLVFQRRQDGSVDFFRSWSSYRAGFGNQESEFWLGNENLHQLTLQGNWELRVELEDFNGNRTFAHYATFRLLGEVDHYQLALGKFSEGTAGDSLSLHSGRPFTTYDADHDSSNSNCAVIVHGAWWYASCYRSNLNGRYAVSEAAAHKYGIDWASGRGVGHPYRRVRMMLR

SEQ ID NO: 3 - DNA, 1059 bp, Homo sapiens
agcaagatggatctactgtggatcctgccctccctgtggcttctcctgcttggggggcctgcctgcctgaagacccaggaacaccccagctgcccaggacccagggaactggaagccagcaaagttgtcctcctgcccagttgtcccggagctccaggaagtcctggggagaagggagccccaggtcctcaagggccacctggaccaccaggcaagatgggccccaagggtgagccaggagatccagtgaacctgctccggtgccaggaaggccccagaaactgccgggagctgttgagccagggcgccaccttgagcggctggtaccatctgtgcctacctgagggcagggccctcccagtcttttgtgacatggacaccgaggggggcggctggctggtgtttcagaggcgccaggatggttctgtggatttcttccgctcttggtcctcctacagagcaggttttgggaaccaagagtctgaattctggctgggaaatgagaatttgcaccagcttactctccagggtaactgggagctgcgggtagagctggaagactttaatggtaaccgtactttcgcccactatgcgaccttccgcctcctcggtgaggtagaccactaccagctggcactgggcaagttctcagagggcactgcaggggattccctgagcctccacagtgggaggccctttaccacctatgacgctgaccacgattcaagcaacagcaactgtgcagtgattgtccacggtgcctggtggtatgcatcctgttaccgatcaaatctcaatggtcgctatgcagtgtctgaggctgccgcccacaaatatggcattgactgggcctcaggccgtggtgtgggccacccctaccgcagggttcggatgatgcttcgatagggcactctggcagccagtgcccttatctctcctgtacagcttccggatcgtcagccaccttgcctttgccaaccacctctgcttgcctgtccacatttaaaaataaaatcattttagccctttcaaaaaaaaaaaaaaaaaaaaaaaaaaaaa